(19) United States
(12) Patent Application Publication
(10) Pub. No.: US 2026/0337548 A1
(43) Pub. Date: Oct. 29, 2026

(54) PARTICLES COMPRISING BIOACTIVE AGENTS

(71) Applicant: FONTERRA CO-OPERATIVE GROUP LIMITED, Auckland (NZ)

(72) Inventors: Timothy Thurlby, Auckland (NZ); Alastair Kenneth Hugh MacGibbon, Auckland (NZ); Tracy Rosalia Coyle, Auckland (NZ); Alan David Welman, Auckland (NZ)

(21) Appl. No.: 19/107,208

(22) PCT Filed: Sep. 1, 2023
(86) PCT No.: PCT/IB2023/058646

§ 371 Date: Feb. 27, 2025

(30) Foreign Application Priority Data
Sep. 1, 2022 (AU) 2022902512

(51) Int. Cl. C12N 1/20; A23L 33/135; C12N 11/04; C12R 1/225

(52) U.S. Cl. CPC C12N 1/20; A23L 33/135; C12N 11/04; C12R 2001/225

(57) ABSTRACT

This invention relates generally to particles comprising a bioactive agent embedded in a biofilm, wherein the biofilm is embedded in a matrix. More specifically, but not exclusively, it relates to particles comprising a biofilm-forming microorganism embedded in a biofilm, wherein the biofilm is embedded in a matrix. Food or beverage products, and methods of increasing the stability of a bioactive agent are also provided.

### FIELD OF THE INVENTION

This invention relates generally to particles comprising a bioactive agent embedded in a biofilm, wherein the biofilm is embedded in a matrix. More specifically, but not exclusively, it relates to particles comprising a biofilm-forming microorganism. Food or beverage products, personal care products, cleaning products, and methods of increasing the stability of a bioactive agent are also provided.

The particles described herein are useful for increasing the shelf life of bioactive agents such as probiotic microorganisms, particularly in high water activity environments at ambient temperatures. These particles are particularly useful in ambient-stable high water activity food and beverage products, such as UHT-treated products, that are stored for extended time periods without refrigeration.

### BACKGROUND

The consumption of products containing bioactive agents is associated with a range of health benefits. Such bioactive agents can include various microorganisms, such as probiotic bacteria.

However, bioactive agents can suffer from limited stability and/or shelf life. For example, when the bioactive agent is a microorganism, a well-known problem in the art is the stability of the microorganism. Microorganisms will tend to become non-viable over time, particularly when incorporated into products with a high water activity. This can make it difficult to provide a product with a high level of active microorganisms.

Furthermore, if the microorganisms in a food or beverage product undergo unrestricted growth, this can lead to alterations in the characteristics of the product, or even spoilage.

Known methods for improving the stability and/or shelf life of bioactive agents typically include storage at low temperatures (such as by refrigeration), or storage in environments with a very low water activity (such as dry powders). It will be appreciated that these methods are not universally applicable, as they are not useful for incorporating bioactive agents into food or beverage products with a high water activity, or products that are to be stored at room temperature.

Therefore, it is desirable to have methods and compositions for increasing the stability and/or shelf life of bioactive agents. This is particularly desirable when the bioactive agents are microorganisms, such as probiotic bacteria; when they are incorporated into a food or beverage product with a high water activity; and when the food or beverage product is to be stored at room temperature for an extended period.

It is an object of the invention to go some way towards addressing one or more of these desiderata, or at least to provide the public with a useful choice.

### SUMMARY OF THE INVENTION

In a first aspect, the invention provides a particle comprising a bioactive agent embedded in a biofilm, wherein the biofilm is embedded in a matrix, wherein the matrix has a water vapour transmission rate (WVTR) from 0.1 to 500.

In a second aspect, the invention provides a particle comprising a bioactive agent embedded in a biofilm, wherein the biofilm is embedded in a matrix comprising at least 6% by weight of one or more lipids.

In a third aspect, the invention provides a food or beverage product comprising the particle of the first or second aspects.

In a fourth aspect, the invention provides a personal care product comprising the particle of the first or second aspects.

In a fifth aspect, the invention provides a cleaning product comprising the particle of the first or second aspects.

In a sixth aspect, the invention provides a method of increasing the stability of a bioactive agent, the method comprising the steps of:
a. embedding the bioactive agent in a biofilm,
b. embedding the biofilm in a matrix having a water vapour transmission rate (WVTR) from 0.1 to 500, and
c. forming the matrix into particles.

In a seventh aspect, the invention provides a method of increasing the stability of a bioactive agent, the method comprising the steps of:
a. embedding the bioactive agent in a biofilm,
b. embedding the biofilm in a matrix comprising at least 6% by weight of one or more lipids, and
c. forming the matrix into particles.

In an eighth aspect, the invention provides a method of increasing the stability of a bioactive agent, the method comprising the steps of:
a. embedding the bioactive agent in a matrix having a water vapour transmission rate (WVTR) from 0.1 to 500,
b. inducing the bioactive agent to produce a biofilm, and
c. forming the matrix into particles,
   wherein steps b) and c) can be in any order.

In some embodiments, step (b) comprises inducing the matrix-embedded bioactive agent to produce a biofilm.

In a ninth aspect, the invention provides a method of increasing the stability of a bioactive agent, the method comprising the steps of:
a. embedding the bioactive agent in a matrix comprising at least 6% by weight of one or more lipids,
b. inducing the bioactive agent to produce a biofilm, and
c. forming the matrix into particles,
   wherein steps b) and c) can be in any order.

In some embodiments, step (b) comprises inducing the matrix-embedded bioactive agent to produce a biofilm.

In some embodiments, the method of the sixth, seventh, eighth, or ninth aspect further comprises the steps of:
d. combining the particles of step c with a medium having a water activity of at least 0.5, preferably a UHT-treated yoghurt, and
e. incubating the medium for between 3 and 30 days.

In a further aspect, the invention provides a method of producing a bioactive agent supplemented food or beverage product, the method comprising the method of the above embodiment, and further comprising the steps of:
f. harvesting the particles from the medium of step e, and
g. combining the harvested particles with a food or beverage product, thus obtaining the bioactive agent supplemented food or beverage product.

In a further aspect, the invention provides a method of producing a bioactive agent supplemented food or beverage product, the method comprising the method of the sixth, seventh, eighth, or ninth aspects, and further comprising the step of combining the particles with a food or beverage product, thus obtaining the bioactive agent supplemented food or beverage product.

The following embodiments can apply to any of the above aspects.

In one embodiment, the matrix comprises at least 6% by weight of one or more lipids.

In one embodiment, the matrix has a water vapour transmission rate (WVTR) from 0.1 to 500 g/m²/24 h, such as from 0.1 to 400, from 0.1 to 300, from 0.1 to 200, from 0.1 to 150, from 0.1 to 100, from 0.1 to 90, from 0.1 to 80, from 0.1 to 70, from 0.1 to 60, from 0.1 to 50, from 0.1 to 40, from 0.1 to 30, from 0.1 to 20, from 0.1 to 10, from 0.1 to 9, from 0.1 to 8, from 0.1 to 7, from 0.1 to 6, from 0.1 to 5, from 0.1 to 4, from 0.1 to 3, from 0.5 to 500, such as from 0.5 to 400, from 0.5 to 300, from 0.5 to 200, from 0.5 to 150, from 0.5 to 100, from 0.5 to 90, from 0.5 to 80, from 0.5 to 70, from 0.5 to 60, from 0.5 to 50, from 0.5 to 40, from 0.5 to 30, from 0.5 to 20, from 0.5 to 10, from 0.5 to 9, from 0.5 to 8, from 0.5 to 7, from 0.5 to 6, from 0.5 to 5, from 0.5 to 4, from 0.5 to 3, from 1 to 500, such as from 1 to 400, from 1 to 300, from 1 to 200, from 1 to 150, from 1 to 100, from 1 to 90, from 1 to 80, from 1 to 70, from 1 to 60, from 1 to 50, from 1 to 40, from 1 to 30, from 1 to 20, from 1 to 10, from 1 to 9, from 1 to 8, from 1 to 7, from 1 to 6, from 1 to 5, from 1 to 4, from 1 to 3, from 2 to 500, such as from 2 to 400, from 2 to 300, from 2 to 200, from 2 to 150, from 2 to 100, from 2 to 90, from 2 to 80, from 2 to 70, from 2 to 60, from 2 to 50, from 2 to 40, from 2 to 30, from 2 to 20, from 2 to 10, from 2 to 9, from 2 to 8, from 2 to 7, from 2 to 6, from 2 to 5, from 2 to 4, from 2 to 3, from 3 to 500, such as from 3 to 400, from 3 to 300, from 3 to 200, from 3 to 150, from 3 to 100, from 3 to 90, from 3 to 80, from 3 to 70, from 3 to 60, from 3 to 50, from 3 to 40, from 3 to 30, from 3 to 20, from 3 to 10, from 3 to 9, from 3 to 8, from 3 to 7, from 3 to 6, from 3 to 5, from 3 to 4, from 4 to 500, such as from 4 to 400, from 4 to 300, from 4 to 200, from 4 to 150, from 4 to 100, from 4 to 90, from 4 to 80, from 4 to 70, from 4 to 60, from 4 to 50, from 4 to 40, from 4 to 30, from 4 to 20, from 4 to 10, from 4 to 9, from 4 to 8, from 4 to 7, from 4 to 6, from 4 to 5, from 5 to 500, such as from 5 to 400, from 5 to 300, from 5 to 200, from 5 to 150, from 5 to 100, from 5 to 90, from 5 to 80, from 5 to 70, from 5 to 60, from 5 to 50, from 5 to 40, from 5 to 30, from 5 to 20, from 5 to 10, from 5 to 9, from 5 to 8, from 5 to 7, or from 5 to 6 g/m²/24 h. In some embodiments, the matrix has a water vapour transmission rate (WVTR) of less than 500 g/m²/24 h, such as less than 400, less than 300, less than 200, less than 150, less than 100, less than 90, less than 80, less than 70, less than 60, less than 50, less than 40, less than 30, less than 20, less than 10, less than 9, less than 8, less than 7, less than 6, less than 5, less than 4, less than 3, less than 2, or about 1 g/m²/24 h.

In one embodiment, the matrix has a water vapour transmission rate (WVTR) from 0.1 to 10, for example from 1 to 5.

In some embodiments, the matrix has a water vapour transmission rate (WVTR) from about 1 to about 350 g/m²/24 h, such as from about 1 to about 325, from about 1 to about 300, from about 1 to about 275, from about 1 to about 250, from about 1 to about 225, from about 1 to about 200, from about 1 to about 175, from about 1 to about 150, from about 1 to about 125, from about 1 to about 100, from about 10 to about 350, from about 10 to about 325, from about 10 to about 300, from about 10 to about 275, from about 10 to about 250, from about 10 to about 225, from about 10 to about 200, from about 10 to about 175, from about 10 to about 150, from about 10 to about 125, from about 10 to about 100, from about 20 to about 350, from about 20 to about 325, from about 20 to about 300, from about 20 to about 275, from about 20 to about 250, from about 20 to about 225, from about 20 to about 200, from about 20 to about 175, from about 20 to about 150, from about 20 to about 125, or from about 20 to about 100 g/m²/24 h.

In some embodiments, the particle has a surface area to volume ratio of from about 1 to about 100, such as from about 1 to about 75, from about 1 to about 50, from about 1 to about 40, from about 1 to about 30, from about 1 to about 20, from about 1 to about 10, from about 1 to about 5, from about 1 to about 3, from about 1 to about 2, from about 1.5 to about 100, from about 1.5 to about 75, from about 1.5 to about 50, from about 1.5 to about 40, from about 1.5 to about 30, from about 1.5 to about 20, from about 1.5 to about 10, from about 1.5 to about 5, from about 1.5 to about 3, or from about 1.5 to about 2.

In some embodiments, the particle has a water activity of from about 0.1 to about 1.0, such as from about 0.1 to about 0.9, from about 0.1 to about 0.8, from about 0.1 to about 0.7, from about 0.1 to about 0.6, from about 0.1 to about 0.5, from about 0.2 to about 1.0, from about 0.2 to about 0.9, from about 0.2 to about 0.8, from about 0.2 to about 0.7, from about 0.2 to about 0.6, from about 0.2 to about 0.5, from about 0.3 to about 1.0, from about 0.3 to about 0.9, from about 0.3 to about 0.8, from about 0.3 to about 0.7, from about 0.3 to about 0.6, from about 0.3 to about 0.5, from about 0.4 to about 1.0, from about 0.4 to about 0.9, from about 0.4 to about 0.8, from about 0.4 to about 0.7, from about 0.4 to about 0.6, or from about 0.4 to about 0.5.

In one embodiment, the bioactive agent comprises a first microorganism.

In one embodiment, the biofilm is produced by the first microorganism.

In one embodiment, the first microorganism is a probiotic microorganism.

In one embodiment, the first microorganism is a Bacillus, Bifidobacterium, Enterococcus, Lacticaseibacillus, Lactiplantibacillus, Lactobacillus, Lactococcus, Ligilactobacillus, Limosilactobacillus, Lentilactobacillus, Saccharomyces, or Streptococcus. In one embodiment, the first microorganism is selected from the group consisting of Bacillus coagulans, Bifidobacterium animalis, Bifidobacterium breve, Bifidobacterium bifidum, Bifidobacterium longum, Lacticaseibacillus casei, Lacticaseibacillus paracasei, Lacticaseibacillus rhamnosus, Lactiplantibacillus plantarum subsp. plantarum, Lactobacillus acidophilus, Lactobacillus delbrueckii, Lactobacillus gasseri, Lactococcus lactis, Lactococcus lactis subsp. lactis, Lactococcus lactis subsp. lactis biovar diacetylactis, Leuconostoc pseudomesenteroides, Ligilactobacillus salivarius, Limosilactobacillus reuteri, Saccharomyces boulardii, and Streptococcus thermophilus; preferably wherein the first microorganism is Lacticaseibacillus rhamnosus HN001. In some embodiments, the first microorganism is Lacticaseibacillus rhamnosus or Lacticaseibacillus paracasei, preferably Lacticaseibacillus rhamnosus HN001 or Lacticaseibacillus paracasei subsp. paracasei IM514.

In one embodiment, the particle comprises the first microorganism in an amount of from 10 to 5×10¹² CFU/g of the particle, such as from 10² to 5×10¹², from 10³ to 5×10¹², from 10⁴ to 5×10¹², from 10⁵ to 5×10¹², from 10⁶ to 5×10¹², from 10⁷ to 5×10¹², from 10⁸ to 5×10¹², from 10⁹ to 5×10¹², from 10¹⁰ to 5×10¹², from 10¹¹ to 5×10¹², from 10¹² to 5×10¹², from 10 to 10¹², from 10² to 10¹², from 10³ to 10¹², 10⁴ to 10¹², 10⁵ to 10¹², 10⁶ to 10¹², from 10⁷ to 10¹², from 10⁸ to 10¹², from 10⁹ to 10¹², from 10¹⁰ to 10¹², from 10¹¹ to 10¹², from 10 to 10¹¹, from 10² to 10¹¹, 10³ to 10¹¹, 10⁴ to 10¹¹, 10⁵ to 10¹¹, from 10⁶ to 10¹¹, from 10⁷ to 10¹¹, from 10⁸ to 10¹¹, from 10⁹ to 10¹¹, from 10¹⁰ to 10¹¹, from 10 to 10¹⁰, from 10² to 10¹⁰, from 10³ to 10¹⁰, from 10⁴ to 10¹⁰, from 10⁵ to 10¹⁰, from 10⁶ to 10¹⁰, from 10⁷ to 10¹⁰, from 10⁸ to 10¹⁰, from 10⁹ to 10¹⁰, from 10 to 10⁹, from 10² to 10⁹, from 10³ to 10⁹, from 10⁴ to 10⁹, from 10⁵ to 10⁹, from 10⁶ to 10⁹, from 10⁷ to 10⁹, from 10⁸ to 10⁹, from 10 to 10⁸, from 10² to 10⁸, from 10³ to 10⁸, from 10⁴ to 10⁸, from 10⁵ to 10⁸, from 10⁶ to 10⁸, from 10⁷ to 10⁸, from 10 to 10⁷, from 10² to 10⁷, from 10³ to 10⁷, from 10⁴ to 10⁷, from 10⁵ to 10⁷, from 10⁶ to 10⁷, from 10 to 10⁶, from 10² to 10⁶, from 10³ to 10⁶, from 10⁴ to 10⁶, from 10⁵ to 10⁶, from 10 to 10⁵, from 10² to 10⁵, from 10³ to 10⁵, from 10⁴ to 10⁵, from 10 to 10⁴, from 10² to 10⁴, from 10³ to 10⁴, from 10 to 10³, from 10² to 10³, or from 10 to 10² CFU/g of the particle.

In one embodiment, the particle further comprises a second microorganism that is different from the first microorganism. In various embodiments, the particle further comprises any number of additional different microorganisms, such as a third, a fourth, and/or a fifth microorganism.

In one embodiment, the second microorganism is a probiotic microorganism. In some embodiments, the third, fourth, and/or fifth microorganism(s) are probiotic microorganism(s). In one embodiment, every microorganism is a probiotic microorganism. In one embodiment, the first microorganism is a biofilm-producing microorganism and the second microorganism is a probiotic microorganism. In another embodiment, the first microorganism is a probiotic microorganism and the second microorganism is a biofilm-producing microorganism.

In one embodiment, the bioactive agent is a non-microbial bioactive agent. In one embodiment, the particle further comprises a non-microbial bioactive agent. In some embodiments, the non-microbial bioactive agent is a protein, a peptide, an amino acid, a fat, a triglyceride, a lipid, a fatty acid, a salt of a fatty acid, an oligosaccharide, a polysaccharide, a nucleic acid, a nucleotide, a nucleoside, a vitamin, a mineral, or any combination of two or more of these. In a preferred embodiment, the non-microbial bioactive agent is a vitamin, a prebiotic, a postbiotic, or a human milk oligosaccharide. In some embodiments, the vitamin is vitamin A, vitamin C, vitamin D, vitamin E, vitamin K, vitamin B1 (thiamine), vitamin B2 (riboflavin), vitamin B3 (niacin), vitamin B6 (pyridoxine), vitamin B12 (cyanocobalamin), vitamin B5 (pantothenic acid), vitamin B7 (biotin), vitamin B9 (folate or folic acid). In some embodiments, the prebiotic is a fructooligosaccharide, a galactooligosaccharide, or an inulin. In some embodiments, the human milk oligosaccharide is 2′-fucosyllactose (2FL), 3′-fucosyllactose (3FL), 3′-sialyllactose (3SL), 6′-sialyllactose (6SL), lacto-N-tetraose (LNT), lacto-N-neotetraose (LNnT), or any combination of two or more of these.

In one embodiment, the biofilm is characterised by peaks in a principal components analysis of Raman spectroscopy data at 360, 430, and/or 530 cm⁻¹. In some embodiments, the Raman spectroscopy data is indicative of the presence of rhamnose, for example by the presence of peaks in a principal components analysis of Raman spectroscopy data at 360, 430, and/or 530 cm⁻¹. In one embodiment, a principal components analysis of Raman spectroscopy data of a biofilm-containing sample has peaks at 360, 430, and/or 530 cm⁻¹ that have peak heights that are higher than corresponding peak heights in a principal components analysis of Raman spectroscopy data of a non-biofilm-containing sample, when normalised by the fat-related peak at 1450 cm⁻¹. In one embodiment, peaks in a principal components analysis of Raman spectroscopy data at 360, 430, and/or 530 cm⁻¹ have a peak height that is at least 50% of a peak height of a fat-related peak at 1450 cm⁻¹, such as at least 55%, at least 60%, at least 65%, at least 70%, at least 75%, at least 80%, at least 85%, at least 90%, at least 95%, or about 100% of a peak height of a fat-related peak at 1450 cm⁻¹, and useful ranges may be selected between any of these values (for example, from 50% to 100%, from 60% to 100%, from 70%, to 100%, from 80% to 100%, from 50% to 90%, from 60% to 90%, from 70%, to 90%, or from 80% to 90%).

In one embodiment, the biofilm is capable of being stained by concanavalin A, wheat germ agglutinin, and/or Ulex europaeus agglutinin. In some embodiments, the biofilm can be detected by concanavalin A, wheat germ agglutinin, and/or Ulex europaeus agglutinin staining that extends beyond bacterial cells.

In one embodiment, the biofilm comprises glucose, galactose, rhamnose, galactosamine, glucosamine, mannose and/or fucose.

In one embodiment, the particle maintains a moisture content of from 1% to 60% by weight within the matrix when included in another foodstuff with a water activity of 1.00 for a period of 2 weeks, preferably a moisture content of from 10% to 60%, more preferably a moisture content of from 20% to 60%.

In one embodiment, the one or more lipids are solid at 5 °C, 10 °C, 15 °C, 20 °C, or 25 °C. In some embodiments, the one or more lipids are solid at 35 °C, such as solid at 34 °C, 33 °C, 32 °C, 31 °C, 30 °C, 29 °C, 28 °C, 27 °C, or 26 °C. In one embodiment, the one or more lipids have a melting temperature of 50 °C or lower, such as 48 °C or lower, 46 °C or lower, 44 °C or lower, 42 °C or lower, 40 °C or lower, 38 °C or lower, 36 °C or lower, 34 °C or lower, 32 °C or lower, or 30 °C or lower. In one embodiment, the one or more lipids have a melting temperature of from 30 °C to 50 °C, such as from 32 °C to 48 °C, from 34 °C to 44 °C, or from 36 °C to 40 °C.

In one embodiment, the one or more lipids comprise, or consist of, mono-, di-, or tri-glycerides. In a preferred embodiment, the one or more lipids comprise, or consist of, triglycerides.

In one embodiment, the one or more lipids comprise C10-C22 fatty acids or salts or esters thereof. In one embodiment, the one or more lipids comprise C12-C20 fatty acids or salts or esters thereof. In one embodiment, the one or more lipids comprise at least one mono-, di-, or tri-glyceride. In a preferred embodiment, the one or more lipids comprise at least one triglyceride. In one embodiment, the at least one mono-, di-, or tri-glyceride comprises at least one C12-C20 fatty acid or a salt or ester thereof. In one embodiment, the at least one mono-, di-, or tri-glyceride comprise at least one fatty acid or ester thereof selected from the group consisting of lauric acid, myristic acid, palmitic acid, stearic acid, and arachidic acid. In one embodiment, each fatty acid or ester thereof of the at least one mono-, di-, or triglyceride is selected from the group consisting of lauric acid, myristic acid, palmitic acid, stearic acid, and arachidic acid. In one embodiment, each fatty acid or ester thereof of the one or more lipids is a C12-C20 fatty acid. In one embodiment, each fatty acid or ester thereof of the one or more lipids is selected from the group consisting of lauric acid, myristic acid, palmitic acid, stearic acid, and arachidic acid.

In some embodiments, the one or more lipids comprise one or more fatty acids and/or salts and/or esters thereof, and at least 1% by weight of the fatty acids and/or salts and/or esters thereof have a carbon chain length of 14 or less, such as at least 2%, 3%, 4%, 5%, 6%, 7%, 8%, 9%, 10%, 12%, 14%, 16%, 18%, 20%, 25%, 30%, 35%, 40%, 45%, 50%, 55%, 60%, 65%, 70%, 75%, or at least 80%, and useful ranges may be selected between any of these values (for example, from 1% to 80%, from 2% to 80%, from 4% to 80%, from 6% to 80%, from 8% to 80%, from 10% to 80%, from 20% to 80%, from 30% to 80%, from 40% to 80%, from 1% to 70%, from 2% to 70%, from 4% to 70%, from 6% to 70%, from 8% to 70%, from 10% to 70%, from 20% to 70%, from 30% to 70%, or from 40% to 70%).

In some embodiments, the one or more lipids comprise fatty acids and/or salts and/or esters thereof, and at least 30% by weight of the fatty acids and/or salts and/or esters thereof have a carbon chain length of 16 or less, such as at least 35%, 40%, 45%, 50%, 55%, 60%, 65%, 70%, 72%, 74%, 76%, 78%, 80%, 82%, 84%, 86%, 88%, or at least 90%, and useful ranges may be selected between any of these values (for example, from 30% to 90%, from 40% to 88%, from 50% to 88%, from 60% to 88%, from 70% to 88%, from 74% to 88%, from 76% to 88%, from 78% to 88%, from 78% to 88%, from 80% to 88%, from 70% to 86% from 74% to 86%, from 76% to 86%, from 78% to 86%, from 78% to 86%, or from 80% to 86%).

In one embodiment, at least 50% by weight of the one or more lipids are saturated fats. In various embodiments, at least 55% by weight of the one or more lipids are saturated fats, such as at least 60%, at least 65%, at least 70%, at least 75%, at least 80%, at least 85%, at least 90%, at least 95%, or at least 99% by weight of the one or more lipids are saturated fats.

In one embodiment, the one or more lipids comprise, or consist of, fully hydrogenated palm kernel stearin, palm stearin, fully hydrogenated coconut oil, hydrogenated palm kernel oil, or a mixture of any two or more of these. Preferably the hydrogenated palm kernel oil is fully hydrogenated.

In some embodiments, the matrix comprises at least 4% by weight of total solids of the one or more lipids, such as at least 8%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% by weight of total solids of the one or more lipids.

In one embodiment, the matrix comprises 4-100% by weight of the one or more lipids, preferably 30-100%. In some embodiments, the matrix comprises from 4% to 100% by weight of total solids of the one or more lipids, such as from 8% to 100%, from 10% to 100%, from 20% to 100%, from 30% to 100%, from 40% to 100%, from 50% to 100%, from 60% to 100%, from 70% to 100%, from 80% to 100%, from 90% to 100%, from 4% to 99.5%, from 8% to 99.5%, from 10% to 99.5%, from 20% to 99.5%, from 30% to 99.5%, from 40% to 99.5%, from 50% to 99.5%, from 60% to 99.5%, from 70% to 99.5%, from 80% to 99.5%, from 90% to 99.5%, from 4% to 95%, from 8% to 95%, from 10% to 95%, from 20% to 95%, from 30% to 95%, from 40% to 95%, from 50% to 95%, from 60% to 95%, from 70% to 95%, from 80% to 95%, from 90% to 95%, from 10% to 90%, from 20% to 90%, from 30% to 90%, from 40% to 90%, from 50% to 90%, from 60% to 90%, from 70% to 90%, from 80% to 90%, from 10% to 80%, from 20% to 80%, from 30% to 80%, from 40% to 80%, from 50% to 80%, from 60% to 80%, from 70% to 80%, from 10% to 70%, from 20% to 70%, from 30% to 70%, from 40% to 70%, from 50% to 70%, from 60% to 70%, from 10% to 60%, from 20% to 60%, from 30% to 60%, from 40% to 60%, from 50% to 60%, from 10% to 50%, from 20% to 50%, from 30% to 50%, from 40% to 50%, from 10% to 40%, from 20% to 40%, from 30% to 40%, from 10% to 20%, from 10% to 30%, or from 10% to 20% by weight of total solids of the one or more lipids.

In one embodiment, the matrix comprises at least 10% by weight of fully hydrogenated coconut oil, preferably at least 15, 20, 25, or 30%.

In one embodiment, the matrix comprises one or more adjuncts selected from sugars, emulsifiers, milk solids, and salts.

In some embodiments, the matrix comprises from 0% to 80% by weight of total solids of one or more sugars, such as from 10% to 80%, from 20% to 80%, from 30% to 80%, from 40% to 80%, from 50% to 80%, from 60% to 80%, from 70% to 80%, from 0% to 70%, from 10% to 70%, from 20% to 70%, from 30% to 70%, from 40% to 70%, from 50% to 70%, from 60% to 70%, from 0% to 60%, from 10% to 60%, from 20% to 60%, from 30% to 60%, from 40% to 60%, from 50% to 60%, from 0% to 50%, from 10% to 50%, from 20% to 50%, from 30% to 50%, from 40% to 50%, from 0% to 40%, from 10% to 40%, from 20% to 40%, from 30% to 40%, from 0% to 30%, from 10% to 30%, from 20% to 30%, from 0% to 20%, from 10% to 20%, or from 0% to 10% by weight of total solids of one or more sugars.

In some embodiments, the matrix comprises from 0% to 2% by weight of total solids of one or more emulsifiers, such as from 0.2% to 2%, from 0.4% to 2%, from 0.6% to 2%, from 0.8% to 2%, from 1.0% to 2%, from 1.2% to 2%, from 1.4% to 2%, from 1.6% to 2%, from 1.8% to 2%, from 0% to 1.8%, from 0.2% to 1.8%, from 0.4% to 1.8%, from 0.6% to 1.8%, from 0.8% to 1.8%, from 1.0% to 1.8%, from 1.2% to 1.8%, from 1.4% to 1.8%, from 1.6% to 1.8%, from 0% to 1.6%, from 0.2% to 1.6%, from 0.4% to 1.6%, from 0.6% to 1.6%, from 0.8% to 1.6%, from 1.0% to 1.6%, from 1.2% to 1.6%, from 1.4% to 1.6%, from 0% to 1.4%, from 0.2% to 1.4%, from 0.4% to 1.4%, from 0.6% to 1.4%, from 0.8% to 1.4%, from 1.0% to 1.4%, from 1.2% to 1.4%, from 0% to 1.2%, from 0.2% to 1.2%, from 0.4% to 1.2%, from 0.6% to 1.2%, from 0.8% to 1.2%, from 1.0% to 1.2%, from 0% to 1.0%, from 0.2% to 1.0%, from 0.4% to 1.0%, from 0.6% to 1.0%, from 0.8% to 1.0%, from 0% to 0.8%, from 0.2% to 0.8%, from 0.4% to 0.8%, from 0.6% to 0.8%, from 0% to 0.6%, from 0.2% to 0.6%, from 0.4% to 0.6%, from 0% to 0.4%, from 0.2% to 0.4%, or from 0% to 0.2% by weight of total solids of one or more emulsifiers.

In some embodiments, the matrix comprises from 0% to 30% milk solids by weight of total solids, such as from 5% to 30%, from 10% to 30%, from 15% to 30%, from 20% to 30%, from 25% to 30%, from 0% to 25%, from 5% to 25%, from 10% to 25%, from 15% to 25%, from 20% to 25%, from 0% to 20%, from 5% to 20%, from 10% to 20%, from 15% to 20%, from 0% to 15%, from 5% to 15%, from 10% to 15%, from 0% to 10%, from 5% to 10%, or from 0% to 5% milk solids by weight of total solids.

In one embodiment, the matrix further comprises by weight of total solids:
a. 20-80% sugars, preferably sucrose and/or lactose;
b. 0.1-20% emulsifiers, preferably sorbitan tristearate and/or lecithin; and/or
c. 10-50% milk solids.

In some embodiments, the matrix comprises by weight of total solids from about 50% to about 70% hydrogenated coconut oil, and from about 30% to about 50% sucrose; more preferably from about 55% to about 65% hydrogenated coconut oil, and from about 35% to about 45% sucrose; most preferably about 59% hydrogenated coconut oil and about 41% sucrose.

In some embodiments, the matrix comprises by weight of total solids from about 20% to about 40% palm stearin, from about 20% to about 40% hydrogenated coconut oil, and from about 30% to about 50% sucrose; preferably from about 25% to about 35% palm stearin, from about 25% to about 35% hydrogenated coconut oil, and from about 35% to about 45% sucrose; most preferably about 29.5% palm stearin, about 29.5% hydrogenated coconut oil, and about 41% sucrose.

In some embodiments, the matrix comprises by weight of total solids from about 0% to about 20% palm stearin, from about 0% to about 20% hydrogenated coconut oil, from about 30% to about 50% hydrogenated palm kernel stearin, and from about 30% to about 50% sucrose; preferably from about 5% to about 15% palm stearin, from about 5% to about 15% hydrogenated coconut oil, from about 35% to about 45% hydrogenated palm kernel stearin, and from about 35% to about 45% sucrose; most preferably about 11% palm stearin, about 11% hydrogenated coconut oil, about 37% hydrogenated palm kernel stearin, and about 41% sucrose.

In some embodiments, the matrix comprises by weight of total solids from about 90% to about 100% cocoa butter, and optionally from about 0.1% to about 1.0% lecithin; preferably from about 95% to about 100% cocoa butter, and optionally from about 0.1% to about 1.0% lecithin; most preferably about 99.5% cocoa butter and about 0.5% lecithin.

In some embodiments, the matrix comprises by weight of total solids from about 65% to about 85% cocoa butter and from about 15% to about 35% milk solids; preferably from about 70% to about 80% cocoa butter and from about 20% to about 30% milk solids; most preferably about 73% cocoa butter and about 26% milk solids.

In some embodiments, the matrix comprises by weight of total solids at least about 80% hydrogenated palm kernel oil, preferably at least about 90%, more preferably at least about 95%, most preferably about 100% hydrogenated palm kernel oil, and useful ranges may be selected between any of these values (for example, from about 80% to about 100%, from about 90% to about 100%, or from about 95% to about 100%).

In some embodiments, the matrix comprises by weight of total solids from about 75% to about 95% cocoa butter and from about 5% to about 25% lactose; preferably from about 80% to about 90% cocoa butter and from about 5% to about 15% lactose; more preferably about 89.5% cocoa butter and about 13.5% lactose.

In some embodiments, the matrix comprises by weight of total solids from about 75% to about 95% hydrogenated coconut oil and from about 5% to about 25% lactose; preferably from about 80% to about 90% hydrogenated coconut oil and from about 10% to about 20% lactose; more preferably about 86.5% hydrogenated coconut oil and about 13.5% lactose.

In one embodiment, the particle has a particle size of from 50 μm to 10 mm, such as from 50 μm to 9 mm, from 50 μm to 8 mm, from 50 μm to 7 mm, from 50 μm to 6 mm, from 50 μm to 5 mm, from 50 μm to 4 mm, from 50 μm to 3 mm, from 50 μm to 2 mm, from 50 μm to 1 mm, from 50 μm to 500 μm, from 100 μm to 10 mm, from 100 μm to 9 mm, from 100 μm to 8 mm, from 100 μm to 7 mm, from 100 μm to 6 mm, from 100 μm to 5 mm, from 100 μm to 4 mm, from 100 μm to 3 mm, from 100 μm to 2 mm, from 100 μm to 1 mm, from 100 μm to 500 μm, from 200 μm to 10 mm, from 200 μm to 9 mm, from 200 μm to 8 mm, from 200 μm to 7 mm, from 200 μm to 6 mm, from 200 μm to 5 mm, from 200 μm to 4 mm, from 200 μm to 3 mm, from 200 μm to 2 mm, from 200 μm to 1 mm, or from 200 μm to 500 μm.

In one embodiment, the particle further comprises at least one coating layer. In some embodiments, the particle comprises two, three, four, or five coating layers. In some embodiments, the coating layer comprises alginate, chitosan, collagen, dextran, pectin, pullulan, gelatin, carrageenan, agar-agar, cellulose, hemicellulose, ethylcellulose, carboxycellulose, or mixtures of any two or more of these.

In one embodiment, the food or beverage product comprises:
a. a protein powder, a protein shake, a protein shot, a protein gel, or a sports nutritional formulation,
b. a UHT beverage, a UHT smoothie,
c. an infant formula, a follow-on formula, a growing-up formula, a paediatric formula, a human milk fortifier, a children's food or drink, a maternal supplement, or a maternal nutritional formulation,
d. a gel such as a heat-set gel, a sauce, a spread, a jam, a jelly, or a honey,
e. an acid-set gel, an ambient stable yoghurt, a stirred yoghurt, a set yoghurt, or a drinking yoghurt,
f. a neutral beverage, an acidic beverage, a water, a juice, a milk, a smoothie, a shake, a shot, an alcoholic beverage, a soft drink, a kombucha, a kefir, or a ready-to-mix powder,
g. a bar, a ball, a cake, a cookie, a muffin, or a bakery good,
h. a medical food, a soup, a dessert, a pudding, a custard, an enteral formulation, a formulation for senior or aged populations, a tablet, a capsule, or a supplement,
i. a confection, a gummy, a candy, a chocolate, a fudge, a truffle, a chewing gum, a frozen dessert, an ice cream
j. flavouring, a topping, or a baking ingredient,
k. a cream, a cheese, or a butter, or
l. a livestock food or a pet food.

In some embodiments, the food or beverage product comprises a yoghurt, a fruit juice such as an orange juice, a sports shake, a cheese sauce, a cream cheese spread, or a milk, for example a non-dairy milk such as an oat milk.

In one embodiment, the food or beverage product has a water activity of at least 0.2, 0.4, 0.6, 0.8, or 0.9.

In one embodiment, the food or beverage product is stable at 30 °C for at least 30 days, preferably for at least 60 days, more preferably for at least 6 months.

In one embodiment, the bioactive agent comprises a microorganism, and the degradation rate of the microorganism measured over 12 months at 30 °C is less than 7 log CFU/g/y, preferably less than 6, 5, 4, 3, 2, or 1 log CFU/g/y.

In one embodiment, the personal care product is a skincare product such as a skin cream, a sunscreen, a haircare product such as a shampoo or a conditioner, a dental product such as a toothpaste or a dentifrice, a deodorant, a cosmetic, a beauty product, or a woman's health product.

In one embodiment of the sixth, seventh, eighth, or ninth aspects, the method further comprises the step of coating the particles in at least one coating layer.

In one embodiment of the method of the sixth, seventh, eighth, or ninth aspects, the particles comprise the particle of the first or second aspects.

In one embodiment of the sixth, seventh, eighth, or ninth aspects, the method further comprises the steps of:
d. combining the particles of step c with a medium having a water activity of at least 0.5, preferably a UHT-treated yoghurt, and
e. incubating the medium for at least 2 weeks.

In one embodiment, the bioactive agent supplemented food or beverage product is a food or beverage product according to the third aspect.

The term “comprising” as used in this specification and claims means “consisting at least in part of”. When interpreting statements in this specification and claims which include the term “comprising”, other features besides the features prefaced by this term in each statement can also be present. Related terms such as “comprise” and “comprised” are to be interpreted in similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE FIGURES

Embodiments of the invention will now be described with reference to the drawings in which:

FIG. 1 shows a contour plot for coating matrices comprising palm stearin, fully hydrogenated palm kernel oil, and fully hydrogenated coconut oil, individually and in combination.

FIG. 2 shows a contour plot for coating matrices comprising palm stearin, fully hydrogenated coconut oil, and cocoa powder, individually and in combination.

FIG. 3 shows a contour plot for coating matrices comprising fully hydrogenated palm kernel oil, fully hydrogenated coconut oil, and cocoa powder, individually and in combination.

FIG. 4 shows a scanning electron microscope image of exopolysaccharide/biofilm-coated Lacticaseibacillus rhamnosus HN001 in a matrix with hydrogenated palm kernel oil at 100× magnification.

FIG. 5 shows a scanning electron microscope image of exopolysaccharide/biofilm-coated Lacticaseibacillus rhamnosus HN001 in a matrix with hydrogenated palm kernel oil at 250× magnification.

FIG. 6 shows a scanning electron microscope image of exopolysaccharide/biofilm-coated Lacticaseibacillus rhamnosus HN001 in a matrix with hydrogenated palm kernel oil at 1,200× magnification.

FIG. 7 shows a scanning electron microscope image of exopolysaccharide/biofilm-coated Lacticaseibacillus rhamnosus HN001 in a matrix with hydrogenated palm kernel oil at 4,000× magnification.

FIG. 8 shows a light microscope image of clusters of Lacticaseibacillus rhamnosus HN001 attached to the surface of a glass wool strand. The image was taken at 100× magnification under oil-immersion and imaged with Crystal Violet stain.

FIG. 9 shows RAMAN spectroscopy of L. rhamnosus HN001 induced to produce biofilm. Three distinct peaks in the 350-600 cm⁻¹ spectral region (indicated with arrows) appear to correlate with the presence of EPS components.

FIG. 10 shows RAMAN spectroscopy of bead structures incorporating L. rhamnosus HN001. A multivariate curve resolution estimated spectrum from analysis of day 120 data from bead sample AT03 is shown. Arrows indicate the same three EPS-related peaks.

FIG. 11 shows concanavalin (Con A) staining of sample MVP17 at day 90 of shelf-life. Mannose/glucose stained with ConA is shown in orange; protein is stained green (this stain will also stain dead cells); cells are stained with DAPI in light blue.

FIG. 12 shows Wheat Germ Agglutinin (WGA) staining of sample MVP17 at day 90 of shelf-life. N-acetylglucosamine stained with WGA is shown in orange; protein is stained green (this stain will also stain dead cells); cells are stained with DAPI in light blue.

FIG. 13 shows Ulex europaeus Agglutinin (UEAI) staining of sample MVP17 at day 90 of shelf-life. UEAI staining is shown in orange; protein is stained green (this stain will also stain dead cells); cells are stained with DAPI in light blue.

### DETAILED DESCRIPTION OF THE INVENTION

In the description in this specification reference may be made to subject matter which is not within the scope of the claims of the current application. That subject matter should be readily identifiable by a person skilled in the art and may assist in putting into practice the invention as defined in the claims of this application.

### Particles

The applicants have surprisingly found that particles comprising a bioactive agent embedded in a biofilm, wherein the biofilm is embedded in a matrix, wherein the matrix has a water vapour transmission rate (WVTR) from 0.1 to 500 provides the bioactive agent with improved shelf-life even when stored at ambient temperatures in high water activity environments.

Accordingly, in a first aspect, the invention provides a particle comprising a bioactive agent embedded in a biofilm, wherein the biofilm is embedded in a matrix, wherein the matrix has a water vapour transmission rate (WVTR) from 0.1 to 500. Preferably, the matrix comprises at least 6% by weight of one or more lipids.

The term “particle” refers to a portion or quantity of material, such as a small portion or quantity of material. The term includes, or may be used interchangeably with, the following: pellet, bead, sphere, granule, etc. In some embodiments, particles may be generally spherical in shape. Preferably, the particle is edible. That is to say, preferably the particle does not comprise any non-edible ingredients.

The term “embedded” as used herein means “at least partly surrounded by”. For example, the bioactive agent is at least partly surrounded by a biofilm, and the biofilm is at least partly surrounded by a matrix. The term is not intended to require that the bioactive agent or the biofilm is entirely surrounded on all sides.

In some embodiments, the bioactive agent is encapsulated within the biofilm. In some embodiments, the biofilm is encapsulated within the matrix. the term “encapsulated within” as used herein means “entirely surrounded by”. In some embodiments, the bioactive agent is dispersed in the biofilm. In some embodiments, the biofilm is dispersed in the matrix.

The term “matrix” refers to a substance or mixture of substances in which biofilm and/or bioactive agent is embedded. The matrix may be any substance or mixture of substances suitable for embedding a bioactive agent and forming a particle. Preferably the matrix is solid at room temperature. In one embodiment, the matrix is edible.

In some embodiments, the matrix comprises one or more lipids, one or more carbohydrates, one or more sugars, one or more proteins, one or more minerals, or any combination of two or more of these.

The term “lipid” refers to a class of organic compounds that are soluble in non-polar solvents. Some examples of lipids include, but are not limited to, fats, fatty acids, waxes, mono-, di-, and tri-glycerides, and phospholipids. Certain lipids, such as fats, may be saturated or unsaturated. Unsaturated lipids may be converted into saturated lipids by the process of hydrogenation. Hydrogenation may be incomplete or partial (if not all carbon-carbon double-bonds are reduced), or may be complete. The term “fully hydrogenated” and related terms such as “completely hydrogenated” and “100% hydrogenated” are intended to mean that at least 99% of the lipid to which the term is applied does not contain an unsaturated carbon-carbon double bond.

In some embodiments, the matrix comprises, consists essentially of, or consists of one or more lipids. Preferably, the one or more lipids are solid at 25 °C.

In some embodiments, the one or more lipids have a melting temperature of 50 °C or lower, such as 48 °C or lower, 46 °C or lower, 44 °C or lower, 42 °C or lower, 40 °C or lower, 38 °C or lower, 36 °C or lower, 34 °C or lower, 32 °C or lower, 30 °C or lower, 25 °C or lower, 20 °C or lower, 15 °C or lower, 10 °C or lower, or 5 °C or lower, and useful ranges may be selected between any of these values (for example, from 5 °C to 50 °C, from 10 °C to 50 °C, from 15 °C to 50 °C, from 20 °C to 50 °C, from 25 °C to 50 °C, from 30 °C to 50 °C, from 32 °C to 50 °C, from 34 °C to 50 °C, from 36 °C to 50 °C, from 5 °C to 48 °C, from 10 °C to 48 °C, from 15 °C to 48 °C, from 20 °C to 48 °C, from 25 °C to 48 °C, from 30 °C to 48 °C, from 32 °C to 48 °C, from 34 °C to 48 °C, from 36 °C to 48 °C, from 5 °C to 46 °C, from 10 °C to 46 °C, from 15 °C to 46 °C, from 20 °C to 46 °C, from 25 °C to 46 °C, from 30 °C to 46 °C, from 32 °C to 46 °C, from 34 °C to 46 °C, from 36 °C to 46 °C, from 5 °C to 44 °C, from 10 °C to 44 °C, from 15 °C to 44 °C, from 20 °C to 44 °C, from 25 °C to 44 °C, from 30 °C to 44 °C, from 32 °C to 44 °C, from 34 °C to 44 °C, from 36 °C to 44 °C, from 5 °C to 42 °C, from 10 °C to 42 °C, from 15 °C to 42 °C, from 20 °C to 42 °C, from 25 °C to 42 °C, from 30 °C to 42 °C, from 32 °C to 42 °C, from 34 °C to 42 °C, from 36 °C to 42 °C, from 5 °C to 40 °C, from 10 °C to 40 °C, from 15 °C to 40 °C, from 20 °C to 40 °C, from 25 °C to 40 °C, from 30 °C to 40 °C, from 32 °C to 40 °C, from 34 °C to 40 °C, or from 36 °C to 40 °C).

In some embodiments, the lipids comprise, consist essentially of, or consist of, triglycerides. In some embodiments, the lipids comprise, consist essentially of, or consist of, C12-C20 fatty acids, or salts or esters thereof.

In some embodiments, the one or more lipids comprise one or more fatty acids and/or salts and/or esters thereof, and at least 1% by weight of the fatty acids and/or salts and/or esters thereof have a carbon chain length of 14 or less, such as at least 2%, 3%, 4%, 5%, 6%, 7%, 8%, 9%, 10%, 12%, 14%, 16%, 18%, 20%, 25%, 30%, 35%, 40%, 45%, 50%, 55%, 60%, 65%, 70%, 75%, or at least 80%, and useful ranges may be selected between any of these values (for example, from 1% to 80%, from 2% to 80%, from 4% to 80%, from 6% to 80%, from 8% to 80%, from 10% to 80%, from 20% to 80%, from 30% to 80%, from 40% to 80%, from 1% to 70%, from 2% to 70%, from 4% to 70%, from 6% to 70%, from 8% to 70%, from 10% to 70%, from 20% to 70%, from 30% to 70%, or from 40% to 70%).

In some embodiments, the one or more lipids comprise fatty acids and/or salts and/or esters thereof, and at least 30% by weight of the fatty acids and/or salts and/or esters thereof have a carbon chain length of 16 or less, such as at least 35%, 40%, 45%, 50%, 55%, 60%, 65%, 70%, 72%, 74%, 76%, 78%, 80%, 82%, 84%, 86%, 88%, or at least 90%, and useful ranges may be selected between any of these values (for example, from 30% to 90%, from 40% to 88%, from 50% to 88%, from 60% to 88%, from 70% to 88%, from 74% to 88%, from 76% to 88%, from 78% to 88%, from 78% to 88%, from 80% to 88%, from 70% to 86% from 74% to 86%, from 76% to 86%, from 78% to 86%, from 78% to 86%, or from 80% to 86%).

In an alternative embodiment, the one or more lipids comprise fatty acids and/or salts and/or esters thereof, and at least 10% by weight of the fatty acids and/or salts and/or esters thereof have a carbon chain length of 16 or less, such as at least 15%, 20%, 25%, 30%, 35%, 40%, 45%, 50%, 55%, 60%, 65%, 70%, 72%, 74%, 76%, 78%, 80%, 82%, 84%, 86%, 88%, or at least 90%, and useful ranges may be selected between any of these values (for example, from 10% to 90%, from 10% to 80%, from 20% to 90%, from 20% to 88%, from 20% to 86%, from 20% to 84%, from 25% to 90%, from 25% to 88%, from 25% to 86%, from 25% to 84%, from 30% to 90%, from 40% to 88%, from 50% to 88%, from 60% to 88%, from 70% to 88%, from 74% to 88%, from 76% to 88%, from 78% to 88%, from 78% to 88%, from 80% to 88%, from 70% to 86% from 74% to 86%, from 76% to 86%, from 78% to 86%, from 78% to 86%, or from 80% to 86%).

Fatty acid esters may include mono-, di-, and/or triglycerides.

In some embodiments, at least 50% by weight of the one or more lipids are saturated fats. In some embodiments, the lipids may be partially or fully hydrogenated. In some embodiments, the one or more lipids comprise, consist essentially of, or consist of, fully hydrogenated palm kernel stearin, palm stearin, fully hydrogenated coconut oil, cocoa butter, or a mixture of any two or more of these. In a preferred embodiment, the one or more lipids comprise, consist essentially of, or consist of, fully hydrogenated palm kernel stearin, palm stearin, fully hydrogenated coconut oil, or a mixture of any two or more of these.

In some embodiments, the matrix comprises at least 4% by weight of total solids of the one or more lipids, such as at least 8%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% by weight of total solids of the one or more lipids. Preferably the matrix comprises at least 30% by weight of total solids of the one or more lipids.

In some embodiments, the matrix comprises from 4% to 95% by weight of total solids of the one or more lipids, such as from 8% to 95%, from 10% to 95%, from 20% to 95%, from 30% to 95%, from 40% to 95%, from 50% to 95%, from 60% to 95%, from 70% to 95%, from 80% to 95%, from 90% to 95%, from 10% to 90%, from 20% to 90%, from 30% to 90%, from 40% to 90%, from 50% to 90%, from 60% to 90%, from 70% to 90%, from 80% to 90%, from 10% to 80%, from 20% to 80%, from 30% to 80%, from 40% to 80%, from 50% to 80%, from 60% to 80%, from 70% to 80%, from 10% to 70%, from 20% to 70%, from 30% to 70%, from 40% to 70%, from 50% to 70%, from 60% to 70%, from 10% to 60%, from 20% to 60%, from 30% to 60%, from 40% to 60%, from 50% to 60%, from 10% to 50%, from 20% to 50%, from 30% to 50%, from 40% to 50%, from 10% to 40%, from 20% to 40%, from 30% to 40%, from 10% to 20%, from 10% to 30%, or from 10% to 20% by weight of total solids of the one or more lipids. Preferably the matrix comprises from 30% to 95% by weight of total solids of the one or more lipids.

In a preferred embodiment, the matrix comprises at least 10% by weight of fully hydrogenated coconut oil, preferably at least 15, 20, 25, or 30%.

In some embodiments, the matrix comprises one or more adjuncts. Examples of adjuncts include sugars, emulsifiers, milk solids, and salts. In some embodiments, adjuncts may be useful for providing additional structural integrity to the particles.

In one embodiment, the matrix comprises, consists essentially of, or consists of hydrogenated palm kernel stearin (preferably fully hydrogenated palm kernel stearin) and a sugar (preferably sucrose and/or lactose). In one embodiment, the matrix comprises, consists essentially of, or consists of hydrogenated palm kernel stearin (preferably fully hydrogenated palm kernel stearin) and milk solids (preferably skim milk powder). In one embodiment, the matrix comprises, consists essentially of, or consists of hydrogenated palm kernel stearin (preferably fully hydrogenated palm kernel stearin), a sugar (preferably sucrose and/or lactose), and milk solids (preferably skim milk powder).

In one embodiment, the matrix comprises, consists essentially of, or consists of palm stearin and a sugar (preferably sucrose and/or lactose). In one embodiment, the matrix comprises, consists essentially of, or consists of palm stearin and milk solids (preferably skim milk powder). In one embodiment, the matrix comprises, consists essentially of, or consists of palm stearin, a sugar (preferably sucrose and/or lactose), and milk solids (preferably skim milk powder).

In one embodiment, the matrix comprises, consists essentially of, or consists of hydrogenated coconut oil (preferably fully hydrogenated coconut oil) and a sugar (preferably sucrose and/or lactose). In one embodiment, the matrix comprises, consists essentially of, or consists of hydrogenated coconut oil (preferably fully hydrogenated coconut oil) and milk solids (preferably skim milk powder). In one embodiment, the matrix comprises, consists essentially of, or consists of hydrogenated coconut oil (preferably fully hydrogenated coconut oil), a sugar (preferably sucrose and/or lactose), and milk solids (preferably skim milk powder).

In one embodiment, the matrix comprises, consists essentially of, or consists of hydrogenated palm kernel oil and a sugar (preferably sucrose and/or lactose). In one embodiment, the matrix comprises, consists essentially of, or consists of hydrogenated palm kernel oil and milk solids (preferably skim milk powder). In one embodiment, the matrix comprises, consists essentially of, or consists of hydrogenated palm kernel oil, a sugar (preferably sucrose and/or lactose), and milk solids (preferably skim milk powder).

In one embodiment, the matrix comprises, consists essentially of, or consists of cocoa butter and a sugar (preferably sucrose and/or lactose). In one embodiment, the matrix comprises, consists essentially of, or consists of cocoa butter and milk solids (preferably skim milk powder). In one embodiment, the matrix comprises, consists essentially of, or consists of cocoa butter, a sugar (preferably sucrose and/or lactose), and milk solids (preferably skim milk powder).

In some embodiments, the matrix comprises from 0% to 80% by weight of total solids of one or more sugars, such as from 10% to 80%, from 20% to 80%, from 30% to 80%, from 40% to 80%, from 50% to 80%, from 60% to 80%, from 70% to 80%, from 0% to 70%, from 10% to 70%, from 20% to 70%, from 30% to 70%, from 40% to 70%, from 50% to 70%, from 60% to 70%, from 0% to 60%, from 10% to 60%, from 20% to 60%, from 30% to 60%, from 40% to 60%, from 50% to 60%, from 0% to 50%, from 10% to 50%, from 20% to 50%, from 30% to 50%, from 40% to 50%, from 0% to 40%, from 10% to 40%, from 20% to 40%, from 30% to 40%, from 0% to 30%, from 10% to 30%, from 20% to 30%, from 0% to 20%, from 10% to 20%, or from 0% to 10% by weight of total solids of one or more sugars. Preferably the matrix comprises from 0% to 50% by weight of total solids of one or more sugars. Preferably the one or more sugars comprise or consist of sucrose and/or lactose.

In some embodiments, the matrix comprises from 0% to 2% by weight of total solids of one or more emulsifiers, such as from 0.2% to 2%, from 0.4% to 2%, from 0.6% to 2%, from 0.8% to 2%, from 1.0% to 2%, from 1.2% to 2%, from 1.4% to 2%, from 1.6% to 2%, from 1.8% to 2%, from 0% to 1.8%, from 0.2% to 1.8%, from 0.4% to 1.8%, from 0.6% to 1.8%, from 0.8% to 1.8%, from 1.0% to 1.8%, from 1.2% to 1.8%, from 1.4% to 1.8%, from 1.6% to 1.8%, from 0% to 1.6%, from 0.2% to 1.6%, from 0.4% to 1.6%, from 0.6% to 1.6%, from 0.8% to 1.6%, from 1.0% to 1.6%, from 1.2% to 1.6%, from 1.4% to 1.6%, from 0% to 1.4%, from 0.2% to 1.4%, from 0.4% to 1.4%, from 0.6% to 1.4%, from 0.8% to 1.4%, from 1.0% to 1.4%, from 1.2% to 1.4%, from 0% to 1.2%, from 0.2% to 1.2%, from 0.4% to 1.2%, from 0.6% to 1.2%, from 0.8% to 1.2%, from 1.0% to 1.2%, from 0% to 1.0%, from 0.2% to 1.0%, from 0.4% to 1.0%, from 0.6% to 1.0%, from 0.8% to 1.0%, from 0% to 0.8%, from 0.2% to 0.8%, from 0.4% to 0.8%, from 0.6% to 0.8%, from 0% to 0.6%, from 0.2% to 0.6%, from 0.4% to 0.6%, from 0% to 0.4%, from 0.2% to 0.4%, or from 0% to 0.2% by weight of total solids of one or more emulsifiers. Preferably the matrix comprises from 0% to 0.5% by weight of total solids of one or more emulsifiers. Preferably the one or more emulsifiers comprise or consist of sorbitan tristearate and/or lecithin, such as soy lecithin.

In some embodiments, the matrix comprises from 0% to 30% milk solids by weight of total solids, such as from 5% to 30%, from 10% to 30%, from 15% to 30%, from 20% to 30%, from 25% to 30%, from 0% to 25%, from 5% to 25%, from 10% to 25%, from 15% to 25%, from 20% to 25%, from 0% to 20%, from 5% to 20%, from 10% to 20%, from 15% to 20%, from 0% to 15%, from 5% to 15%, from 10% to 15%, from 0% to 10%, from 5% to 10%, or from 0% to 5% milk solids by weight of total solids. Preferably the matrix comprises from 20% to 30% milk solids by weight of total solids.

In a preferred embodiment, the matrix comprises by weight of total solids: 20-80% sugars, preferably sucrose; 0.1-20% emulsifiers, preferably sorbitan tristearate and/or lecithin; and/or 10-50% milk solids.

In some embodiments, the particle further comprises an additional coating layer. Such layers may be used to modify the permeability of the particle, such as the water permeability, water vapour transmission rate, or diffusivity. Alternatively or additionally, coating layers may be used to provide resistance to environmental conditions such as resistance to gastric juices.

In some embodiments, the coating layer comprises agar-agar, alginate, carboxycellulose, carrageenan, cellulose, cellulose acetate phthalate, chitosan, collagen, dextran, ethylcellulose, gelatin, gluco-mannans, hemicellulose, milk proteins, shellac, starch, pectin, poly-L-lysine, pullulan, or mixtures of any two or more of these.

In some embodiments, the matrix is solid at 25 °C.

In some embodiments, the matrix has a melting temperature of at least about 30 °C, such as at least about 32 °C, 34 °C, 35 °C, 36 °C, 38 °C, 40 °C, 42 °C, 44 °C, 45 °C, 46 °C, 48 °C, 50 °C, 52 °C, 54 °C, or at least about 55 °C, and useful ranges may be selected between any of these values (for example, from about 30 °C to about 55 °C, from about 35 °C to about 50 °C, or from about 40 °C to about 48 °C).

The particles can be made any suitable size using conventional techniques. It will be appreciated that different applications may require different sized particles. Particle size may be measured using a variety of techniques known in the art. The technique used to measure particle size may vary depending on the size of the particles to be measured. For example, in one embodiment, particle size is measured by microscopy. This is particularly useful for particles ≥1 mm in diameter. In another embodiment, particle size is measured using a Malvern Mastersizer 3000 or similar analyser. This is particularly useful for measuring particles <1 mm in diameter.

In some embodiments, the particles have a diameter of from 50 μm to 10 mm, such as from 50 μm to 9 mm, from 50 μm to 8 mm, from 50 μm to 7 mm, from 50 μm to 6 mm, from 50 μm to 5 mm, from 50 from μm to 4 mm, from 50 μm to 3 mm, from 50 μm to 2 mm, from 50 μm to 1 mm, from 50 μm to 900 μm, from 50 μm to 800 μm, from 50 μm to 700 μm, from 50 μm to 600 μm, from 50 μm to 500 μm, from 50 μm to 400 μm, from 50 μm to 300 μm, from 50 μm to 200 μm, from 75 μm to 10 mm, from 75 μm to 9 mm, from 75 μm to 8 mm, from 75 μm to 7 mm, from 75 μm to 6 mm, from 75 μm to 5 mm, from 75 from μm to 4 mm, from 75 μm to 3 mm, from 75 μm to 2 mm, from 75 μm to 1 mm, from 75 μm to 900 μm, from 75 μm to 800 μm, from 75 μm to 700 μm, from 75 μm to 600 μm, from 75 μm to 500 μm, from 75 μm to 400 μm, from 75 μm to 300 μm, from 75 μm to 200 μm, from 100 μm to 10 mm, from 100 μm to 9 mm, from 100 μm to 8 mm, from 100 μm to 7 mm, from 100 μm to 6 mm, from 100 μm to 5 mm, from 100 from μm to 4 mm, from 100 μm to 3 mm, from 100 μm to 2 mm, from 100 μm to 1 mm, from 100 μm to 900 μm, from 100 μm to 800 μm, from 100 μm to 700 μm, from 100 μm to 600 μm, from 100 μm to 500 μm, from 100 μm to 400 μm, from 100 μm to 300 μm, from 100 μm to 200 μm, from 200 μm to 10 mm, from 200 μm to 9 mm, from 200 μm to 8 mm, from 200 μm to 7 mm, from 200 μm to 6 mm, from 200 μm to 5 mm, from 200 from μm to 4 mm, from 200 μm to 3 mm, from 200 μm to 2 mm, from 200 μm to 1 mm, from 200 μm to 900 μm, from 200 μm to 800 μm, from 200 μm to 700 μm, from 200 μm to 600 μm, from 200 μm to 500 μm, from 200 μm to 400 μm, or from 200 μm to 300 μm.

The applicants have found that particles of the invention are not completely impervious to the ingress of moisture and/or other compounds present in their environment. The applicants have surprisingly found that, contrary to prior art teachings, bioactive agents such as microorganisms are able to be maintained at high viability within the particles of the invention for extended periods, even when stored at ambient temperature in high water activity environments.

Therefore, in some embodiments, the particles are not impervious to moisture. Accordingly, in some embodiments, the matrix has a water vapour transmission rate (WVTR) from 0.1 to 500, such as from 0.1 to 400, from 0.1 to 300, from 0.1 to 200, from 0.1 to 150, from 0.1 to 100, from 0.1 to 90, from 0.1 to 80, from 0.1 to 70, from 0.1 to 60, from 0.1 to 50, from 0.1 to 40, from 0.1 to 30, from 0.1 to 20, from 0.1 to 10, from 0.1 to 9, from 0.1 to 8, from 0.1 to 7, from 0.1 to 6, from 0.1 to 5, from 0.1 to 4, from 0.1 to 3, from 0.5 to 500, such as from 0.5 to 400, from 0.5 to 300, from 0.5 to 200, from 0.5 to 150, from 0.5 to 100, from 0.5 to 90, from 0.5 to 80, from 0.5 to 70, from 0.5 to 60, from 0.5 to 50, from 0.5 to 40, from 0.5 to 30, from 0.5 to 20, from 0.5 to 10, from 0.5 to 9, from 0.5 to 8, from 0.5 to 7, from 0.5 to 6, from 0.5 to 5, from 0.5 to 4, from 0.5 to 3, from 1 to 500, such as from 1 to 400, from 1 to 300, from 1 to 200, from 1 to 150, from 1 to 100, from 1 to 90, from 1 to 80, from 1 to 70, from 1 to 60, from 1 to 50, from 1 to 40, from 1 to 30, from 1 to 20, from 1 to 10, from 1 to 9, from 1 to 8, from 1 to 7, from 1 to 6, from 1 to 5, from 1 to 4, from 1 to 3, from 2 to 500, such as from 2 to 400, from 2 to 300, from 2 to 200, from 2 to 150, from 2 to 100, from 2 to 90, from 2 to 80, from 2 to 70, from 2 to 60, from 2 to 50, from 2 to 40, from 2 to 30, from 2 to 20, from 2 to 10, from 2 to 9, from 2 to 8, from 2 to 7, from 2 to 6, from 2 to 5, from 2 to 4, from 2 to 3, from 3 to 500, such as from 3 to 400, from 3 to 300, from 3 to 200, from 3 to 150, from 3 to 100, from 3 to 90, from 3 to 80, from 3 to 70, from 3 to 60, from 3 to 50, from 3 to 40, from 3 to 30, from 3 to 20, from 3 to 10, from 3 to 9, from 3 to 8, from 3 to 7, from 3 to 6, from 3 to 5, from 3 to 4, from 4 to 500, such as from 4 to 400, from 4 to 300, from 4 to 200, from 4 to 150, from 4 to 100, from 4 to 90, from 4 to 80, from 4 to 70, from 4 to 60, from 4 to 50, from 4 to 40, from 4 to 30, from 4 to 20, from 4 to 10, from 4 to 9, from 4 to 8, from 4 to 7, from 4 to 6, from 4 to 5, from 5 to 500, such as from 5 to 400, from 5 to 300, from 5 to 200, from 5 to 150, from 5 to 100, from 5 to 90, from 5 to 80, from 5 to 70, from 5 to 60, from 5 to 50, from 5 to 40, from 5 to 30, from 5 to 20, from 5 to 10, from 5 to 9, from 5 to 8, from 5 to 7, or from 5 to 6, In some embodiments, the matrix has a water vapour transmission rate (WVTR) of less than 500, such as less than 400, less than 300, less than 200, less than 150, less than 100, less than 90, less than 80, less than 70, less than 60, less than 50, less than 40, less than 30, less than 20, less than 10, less than 9, less than 8, less than 7, less than 6, less than 5, less than 4, less than 3, less than 2, or about 1.

Water vapour transmission rate (WVTR) is a measure of the amount of water vapour transmitted through unit area of test specimen per unit time under specified conditions. Water vapor transmission rate is expressed in grams per square meter per day (g·m⁻²·day⁻¹ or g/m²/24 h). WVTR can be measured using techniques known in the art, for example using a Versaperm MkV Digital WVTR Meter of (Versaperm Ltd., Maidenhead, UK) with 10 cm² measuring area, equipped with an electrolytic detection sensor. In one embodiment, the method of measuring WVTR is based on ISO 15106-3:2003. In a preferred embodiment, WVTR is measured at about 20 °C and about 75% relative humidity for 24 hours. Relative humidity may be maintained at about 75% by use of an open container containing a saturated NaCl solution.

Alternatively, WVTR can be calculated using the following equation:

WVTR=dm/(A·dt)

where dm=the mass of the absorbed water during time dt, and A=total surface area of samples calculated based on particle size measurements. The mass gain should be measured during the period of approximately linear weight gain, for example over a period of 5 minutes, preferably 7 minutes after adjusting the relative humidity to 95%. WVTR is reported in units of g/m²/24 h.

To measure WVTR, an aliquot of the particle sample can be weighed in an intrinsic sorption microbalance of a dynamic vapor sorption (DVS) instrument (Surface Measurement Systems, Alperton, Middlesex, UK), with measurement performed at 95% relative humidity.

In some embodiments, the particles maintain a moisture content of from 1 to 60% by weight within the matrix when included in another foodstuff with a water activity of from 0.90 to 1.00 for a period of 2 weeks, preferably a moisture content of from 10% to 60%, more preferably a moisture content of from 20% to 60%. Moisture content can be measured by a variety of techniques, such as by Raman spectroscopy.

In some embodiments, when the bioactive agent is a microorganism, the microorganism produces a biofilm. Without wishing to be bound by theory, it is believed that the physical and/or chemical properties of the particles, including when the particles are present in a food or beverage product, may induce the microorganism to produce a biofilm. This may be the result of the maintenance of a non-zero moisture content within the matrix, and/or the results of influx of other compounds from the environment, for example lactose.

In some embodiments, the composition of the matrix is selected to favour biofilm formation. For example, in some embodiments, the matrix may include components known to stimulate a microorganism to produce biofilm. In some embodiments, the matrix composition is selected to provide conditions, such as moisture and/or nutrient ingress, that stimulate a microorganism to produce biofilm.

In some embodiments, the particle further comprises at least one coating layer. Coating layers can provide the particle with improved or altered properties, such as increased structural integrity. Coating layers can also be used to alter the water permeability of the particles. In some embodiments, multiple coating layers may be used. Preferably the coating layer is edible.

Some exemplary coating layers include certain polysaccharides including cellulose and its derivatives such as cellulose acetate phthalate, hemicellulose, methyl cellulose, ethyl cellulose, carboxycellulose, carboxymethyl cellulose (CMC), hydroxyethyl cellulose (HEC), hydroxypropyl cellulose, hydroxypropyl methylcellulose (HPMC), and microcrystalline cellulose; lipids and resins including waxes and oils such as paraffin wax, carnauba wax, beeswax, candelilla wax, and polyethylene wax; fatty acids and monoglycerides such as stearyl alcohol, stearic acid, palmitic acid, mono-, di- and tri-glycerides; naturally-occurring resins such as wood resin and coumarone-indene; proteins including corn zein (α-zein, β-zein and/or γ-zein), milk proteins, wheat gluten, soy protein, peanut protein, keratin, collagen, gelatin, casein, and whey proteins; starches and derivatives such as raw starch, modified starch, pregelatinized starch, dextrin, dextran, maltodextrin, corn syrup sucrose, dextrose/fructose and sugar polyols; extrudate gums such as gum arabic, gum ghatti, gum karaya and gum tragacanth; seed gums such as guar gum and locust bean gum; microbial fermentation gums such as xanthan, gellan gum and chitosan; seaweed extracts such as agar, alginates, carageenans and furcellaran; pectins; gluco-mannans; shellac; and poly-L-lysine. Mixtures of these materials can also be used.

The coating layer may also include one or more plasticisers. Suitable plasticisers include glycols such as polyethylene glycol (PEG), polypropylene glycol (PPG), etc., lipids such as vegetable oils, mineral oils, medium chain triglycerides, fats, fatty acids, waxes, etc.

In one embodiment, the particle is encapsulated in a liposphere. In one embodiment, the biofilm is encapsulated in a liposphere which is embedded in the matrix.

### Bioactive Agents

The term “bioactive agent” refers to any material with biological activity, including but not limited to a protein, a peptide, an amino acid, a fat, a triglyceride, a lipid, a fatty acid, an oligosaccharide, a polysaccharide, a nucleic acid, a nucleotide, a nucleoside, a vitamin, a mineral, a microorganism, a derivative of a microorganism, or any combination of two or more of these. It will be understood that the biological activity need not apply directly to an organism that has consumed the particles of the invention. For example, the bioactive agent may comprise a material that has an effect on the gut microbiota of an organism, such as a prebiotic.

In a preferred embodiment, the bioactive agent is a microorganism, more preferably a probiotic microorganism, most preferably a probiotic bacterium.

In some embodiments, the bioactive agent is a derivative of a microorganism, preferably a probiotic microorganism. A derivative of a microorganism can include a mutant or homologue of a microorganism, an attenuated or killed microorganism, and/or a component of a microorganism that still has useful activity. For example, while the bacterial molecules responsible for mediating probiotic activity have not been clearly identified, molecules that have been proposed as possible candidates include bacterial DNA motifs, surface proteins, small organic acids, polysaccharides, and cell wall components such as lipoteichoic acids and peptidoglycan. It has been postulated that these interact with components of the host immune system to give an immuno-modulatory effect. Preferably, the retained activity is at least about 35, 40, 45, 50, 55, 60, 65, 70, 75, 80, 85, 90, 95, 99 or 100% of the activity of an untreated (i.e., live or non-attenuated) control, and useful ranges may be selected between any of these values (for example, from about 35 to about 100%, from about 50 to about 100%, from about 60 to about 100%, from about 70 to about 100%, from about 80 to about 100%, and from about 90 to about 100%).

In some embodiments, the bioactive agent is a non-microbial bioactive agent. The term “non-microbial” means that the bioactive agent does not comprise a live microorganism.

In some embodiments, the non-microbial bioactive agent does not comprise a live or a killed microorganism. In some embodiments, the non-microbial bioactive agent does not comprise a microorganism derivative. In some embodiments, the non-microbial bioactive agent is a protein, a peptide, an amino acid, a fat, a triglyceride, a lipid, a fatty acid, an oligosaccharide, a polysaccharide, a nucleic acid, a nucleotide, a nucleoside, a vitamin, a mineral, or any combination of two or more of these. In a preferred embodiment, the non-microbial bioactive agent is a vitamin, a prebiotic, a postbiotic, or a human milk oligosaccharide. In some embodiments, the vitamin is vitamin A, vitamin C, vitamin D, vitamin E, vitamin K, vitamin B1 (thiamine), vitamin B2 (riboflavin), vitamin B3 (niacin), vitamin B6 (pyridoxine), vitamin B12 (cyanocobalamin), vitamin B5 (pantothenic acid), vitamin B7 (biotin), vitamin B9 (folate or folic acid). In some embodiments, the prebiotic is a fructooligosaccharide, a galactooligosaccharide, or an inulin. In some embodiments, the human milk oligosaccharide is 2′-fucosyllactose (2FL), 3′-fucosyllactose (3FL), 3′-sialyllactose (3SL), 6′-sialyllactose (6SL), lacto-N-tetraose (LNT), lacto-N-neotetraose (LNnT), or any combination of two or more of these.

A prebiotic is a compound that induces the growth or activity of beneficial microorganisms. Some prebiotics are complex carbohydrates that are non-digestible by the organism (e.g. the mammal) that ingested them, but are digestible by beneficial microorganisms. In this manner, some prebiotics can be used as a nutrient source by the beneficial microorganisms. Some non-limiting examples of prebiotics include fructooligosaccharides, galactooligosaccharides, and inulin.

A postbiotic is a product from a microorganism that provides benefit(s) to the host. Some postbiotics are secreted by live micoorganisms, and others are released after bacterial lysis. Some non-limiting examples of postbiotics include vitamins such as vitamins B and K, short-chain fatty acids, and antimicrobial peptides.

### Microorganisms

It is anticipated that the particles and methods of the invention can be useful to provide improved stability and/or shelf life to a wide variety of microorganisms.

In one embodiment, the microorganism is a probiotic microorganism. In a preferred embodiment, the microorganism is a probiotic bacterium. In an alternative embodiment, the microorganism is a fungus, such as a yeast. In one embodiment, the microorganism is in a reproductively viable form.

The term “probiotic” refers to a microorganism possessing probiotic activity. The term “probiotic activity” refers to the ability of certain microorganisms to stimulate the immune system. Measuring the type and level of activity of a probiotic microorganism is known to those skilled in the art; see, for example, Mercenier et al. (2004), Leyer et al. (2004), or Cummings et al. (2004). Preferably probiotic activity is assessed by a PBMC cytokine secretion assay.

In one embodiment, the microorganism is a lactic acid bacterium (LAB). In one embodiment, the microorganism is selected from Bacillus coagulans, Bifidobacterium animalis (for example, Bifidobacterium animalis subsp. lactis strain HN019 or Bifidobacterium animalis subsp. lactis strain BB12), Bifidobacterium breve, Bifidobacterium bifidum, Bifidobacterium longum, Lacticaseibacillus casei (formerly Lactobacillus casei), Lacticaseibacillus paracasei (formerly Lactobacillus paracasei), Lacticaseibacillus rhamnosus (formerly Lactobacillus rhamnosus; for example L. rhamnosus HN001), Lactiplantibacillus plantarum subsp. plantarum (formerly Lactobacillus plantarum), Lactobacillus acidophilus (for example, Lactobacillus acidophilus (LAVRI-A1)), Lactobacillus delbrueckii, Lactobacillus gasseri, Lactococcus lactis, Lactococcus lactis subsp. lactis, Lactococcus lactis subsp. lactis biovar diacetylactis, Leuconostoc pseudomesenteroides, Ligilactobacillus salivarius (formerly Lactobacillus salivarius), Limosilactobacillus reuteri (formerly Lactobacillus reuteri; for example L. reuteri ATCC 55730), Saccharomyces boulardii, Streptococcus thermophilus, or a combination of any two or more thereof.

Preferably, the microorganism is a biofilm-forming microorganism. The ability of a microorganism to form a biofilm can be assessed by means known in the art. For example, microorganisms may be grown in the presence of glass fibres or an activated carbon surface and biofilm formation determined by their adherence to the substrate as described in Example 4.

In some embodiments, the particle comprises a second microorganism that is different from the first microorganism. In some embodiments the second microorganism is a probiotic microorganism. In some embodiments the second microorganism is selected from any of those listed above. Further bioactive agents and/or microorganisms may also be included, for example a third and/or a fourth microorganism.

Combinations of microorganisms may be chosen to have complementary properties. For example, a biofilm-producing microorganism may be combined with a non-producing microorganism. In some embodiments, a microorganism that is a prolific producer of biofilm may be combined with microorganism that is a less prolific producer of biofilm. In some embodiments, the particle comprises a first and a second microorganism, wherein the biofilm is produced by the first microorganism.

One or more microorganisms may also be combined with one or more non-microbial bioactive agents. For example, in some embodiments the bioactive agent comprises a first microorganism, and the particle further comprises a non-microbial bioactive agent. Preferably the non-microbial bioactive agent is different from any component or product of the first microorganism.

In some embodiments, the particle comprises the first and/or the second microorganism in an amount of from 10 to 5×10¹² CFU/g of the particle, such as from 10² to 5×10¹², from 10³ to 5×10¹², from 10⁴ to 5×10¹², from 10⁵ to 5×10¹², from 10⁶ to 5×10¹², from 10⁷ to 5×10¹², from 10⁸ to 5×10¹², from 10⁹ to 5×10¹², from 10¹⁰ to 5×10¹², from 10¹¹ to 5×10¹², from 10¹² to 5×10¹², from 10 to 10¹², from 10² to 10¹², from 10³ to 10¹², 10⁴ to 10¹², 10⁵ to 10¹², 10⁶ to 10¹², from 10⁷ to 10¹², from 10⁸ to 10¹², from 10⁹ to 10¹², from 10¹⁰ to 10¹², from 10¹¹ to 10¹², from 10 to 10¹¹, from 10² to 10¹¹, 10³ to 10¹¹, 10⁴ to 10¹¹, 10⁵ to 10¹¹, from 10⁶ to 10¹¹, from 10⁷ to 10¹¹, from 10⁸ to 10¹¹, from 10⁹ to 10¹¹, from 10¹⁰ to 10¹¹, from 10 to 10¹⁰, from 10² to 10¹⁰, from 10³ to 10¹⁰, from 10⁴ to 10¹⁰, from 10⁵ to 10¹⁰, from 10⁶ to 10¹⁰, from 10⁷ to 10¹⁰, from 10⁸ to 10¹⁰, from 10⁹ to 10¹⁰, from 10 to 10⁹, from 10² to 10⁹, from 10³ to 10⁹, from 10⁴ to 10⁹, from 10⁵ to 10⁹, from 10⁶ to 10⁹, from 10⁷ to 10⁹, from 10⁸ to 10⁹, from 10 to 10⁸, from 10² to 10⁸, from 10³ to 10⁸, from 10⁴ to 10⁸, from 10⁵ to 10⁸, from 10⁶ to 10⁸, from 10⁷ to 10⁸, from 10 to 10⁷, from 10² to 10⁷, from 10³ to 10⁷, from 10⁴ to 10⁷, from 10⁵ to 10⁷, from 10⁶ to 10⁷, from 10 to 10⁶, from 10² to 10⁶, from 10³ to 10⁶, from 10⁴ to 10⁶, from 10⁵ to 10⁶, from 10 to 10⁵, from 10² to 10⁵, from 10³ to 10⁵, from 10⁴ to 10⁵, from 10 to 10⁴, from 10² to 10⁴, from 10³ to 10⁴, from 10 to 10³, from 10² to 10³, or from 10 to 10² CFU/g of the particle.

### Biofilms

The term “biofilm” refers to a layer of extracellular material produced by, and encompassing, microorganisms.

Biofilms are produced by certain microorganisms, such as bacteria, in response to certain environmental conditions. The conditions required to stimulate biofilm production may vary depending on the microorganism, but in some species biofilm production can be induced by stress. Such stresses can include nutrient limitation, osmotic stress, desiccation, exposure to UV light, unfavourable pH or temperature, high pressure, or exposure to certain chemical compounds, including antimicrobials such as antibiotics, and compounds involved in quorum sensing.

Biofilms typically comprise a mucilaginous extracellular layer that encompasses the microorganisms. The composition of a biofilm may differ depending on the species of microorganism that produced it. The major component of bacterial biofilms is typically high molecular weight extracellular microbial polymers such as exopolysaccharides (EPS). It will be appreciated that the term “exopolysaccharide” or “EPS” is a general description that covers a wide range of different compounds with varying chemical and physical properties.

Biofilms provide bacteria with a number of benefits, including increased ability to adhere to a surface, and increased resistance to stressors.

Without wishing to be bound by theory, it is believed that the stability and/or shelf life of a bioactive agent can be improved by embedding the bioactive agent in a biofilm, wherein the biofilm is embedded in a matrix.

Biofilms can be detected by a number of methods known in the art. Such methods include Raman spectroscopy, scanning electron microscopy (SEM), and lectin staining. Some exemplary methods of detecting biofilms are presented in Examples 5, 6 and 8.

In some embodiments, the biofilm can be detected by Raman spectroscopy. Using Raman spectroscopy, hyperspectral maps can be generated that provide structural information on the location of components of biofilm within the scanned area of the sample. In terms of biofilm identification, this provides contextual information on where the bacterial cells are and if there are indeed exopolymeric components around them indicative of biofilm.

In one embodiment, the biofilm is characterised by peaks in a principal components analysis of Raman spectroscopy data at 360, 430, and/or 530 cm⁻¹. In some embodiments, the Raman spectroscopy data is indicative of the presence of rhamnose, for example by the presence of peaks in a principal components analysis of Raman spectroscopy data at 360, 430, and/or 530 cm⁻¹. In one embodiment, peaks in a principal components analysis of Raman spectroscopy data at 360, 430, and/or 530 cm⁻¹ have a peak height that is at least 50% of a peak height of a fat-related peak at 1450 cm⁻¹, such as at least 55%, at least 60%, at least 65%, at least 70%, at least 75%, at least 80%, at least 85%, at least 90%, at least 95%, or about 100% of a peak height of a fat-related peak at 1450 cm⁻¹, and useful ranges may be selected between any of these values (for example, from 50% to 100%, from 60% to 100%, from 70%, to 100%, from 80% to 100%, from 50% to 90%, from 60% to 90%, from 70%, to 90%, or from 80% to 90%).

For example, exopolysaccharide (EPS) produced by Lacticaseibacillus rhamnosus HN001 contains signature sugars of glucose, galactose and rhamnose, with trace amounts of galactosamine, glucosamine, mannose and fucose. Because residual glucose and galactose may be present in growth media and/or food or beverage compositions, identification of rhamnose, galactosamine, glucosamine, mannose and fucose can provide strong evidence of the presence of biofilm associated with L. rhamnosus HN001 in the particles of the invention. In some embodiments, the biofilm comprises glucose, galactose, rhamnose, galactosamine, glucosamine, mannose and/or fucose.

In some embodiments, the biofilm can be detected by lectin staining. Lectin staining can be used to detect the presence of sugars present in L. rhamnosus HN001 EPS, such as glucose, galactose, rhamnose, galactosamine, glucosamine, mannose and fucose. For example, biofilm formation can be detected by fluorescence microscopy using fluorescein-linked lectins. The present inventors have demonstrated that the presence of mannose, N-acetylglucosamine and fucose provides strong evidence of the presence of biofilm associated with L. rhamnosus HN001 in the particles of the invention.

To detect biofilm by lectin staining, a particle can be cut into sections about 2 mm thick. The cut sections are stained with 5 μl of 0.1 mg/ml fluorescein tagged lectin stain, 1 μM DAPI and 0.2% fast green. Different lectin stains can be used to detect different sugars. In one embodiment, the lectin stains may be selected from concanavalin A, wheat germ agglutinin, and/or Ulex europaeus agglutinin. The samples are allowed to stain for at least 15 minutes before imaging using an inverted confocal fluorescence microscope. A 407 nm laser is used to excite DAPI, a 488 nm laser is used to excite fluorescein-tagged lectins, and a 561 nm excitation is used for fast green. The presence of bacterial cells embedded in biofilm can be determined by co-localisation of cells stained with DAPI stain, and biofilm stained with the one or more lectins. Various methods for detecting biofilm by lectin staining are presented in Examples 6, 8 and 11.

In some embodiments, the biofilm is produced by the bioactive agent. For example, in some embodiments, the bioactive agent is a first microorganism, and the biofilm is produced by the first microorganism. In some embodiments, the first microorganism is embedded in the matrix, wherein it produces the biofilm. In other embodiments, the first microorganism is induced to produce the biofilm, which is then embedded in the matrix. The first microorganism may be induced to produce the biofilm by exposure to stresses such as nutrient limitation, osmotic stress, desiccation, exposure to UV light, unfavourable pH or temperature, high pressure, or exposure to certain chemical compounds, including antimicrobials such as antibiotics, and compounds involved in quorum sensing.

In some embodiments, the particle further comprises a second microorganism that is different from the first microorganism. In some embodiments, the second microorganism is a probiotic microorganism. This may be useful, for example, where the first microorganism is included for the purposes of biofilm production, and where the second microorganism is included for the purposes of providing a benefit, such as a health benefit. Accordingly, in some embodiments the particle comprises a first and a second microorganism, wherein the biofilm is produced by the first microorganism.

### Food and Beverage Products

In a second aspect, the invention provides a food or beverage product comprising the particle of the first aspect.

The term “food or beverage product” refers to any edible and/or drinkable product or ingredient. The term is intended to include products for human consumption and products for non-human consumption, such as a pet food or a livestock food. The term is also intended to include products that must undergo further processing before being in a consumable form, for example a drink powder that is dissolved in a liquid to form a beverage. It will be appreciated that in some embodiments, the food or beverage product will be an ingredient for incorporation into other products, such as a bakery ingredient.

It is anticipated that the particles of the invention allow the production of food and/or beverage products with improved stability and/or shelf life.

In some embodiments, the food or beverage product comprises a protein powder, a protein shake, a protein shot, a protein gel, or a sports nutritional formulation, a UHT beverage, a UHT smoothie, an infant formula, a follow-on formula, a growing-up formula, a paediatric formula, a human milk fortifier, a children's food or drink, a maternal supplement, or a maternal nutritional formulation, a gel such as a heat-set gel, a sauce, a spread, a jam, a jelly, or a honey, an acid-set gel, an ambient stable yoghurt, a stirred yoghurt, a set yoghurt, or a drinking yoghurt, a neutral beverage, an acidic beverage, a water, a juice, a milk, a smoothie, a shake, a shot, an alcoholic beverage, a soft drink, a kombucha, a kefir, or a ready-to-mix powder, a bar, a ball, a cake, a cookie, a muffin, or a bakery good, a medical food, a soup, a dessert, a pudding, a custard, an enteral formulation, a formulation for senior or aged populations, a tablet, a capsule, or a supplement, a confection, a gummy, a candy, a chocolate, a fudge, a truffle, a chewing gum, a frozen dessert, an ice cream, flavouring, a topping, or a baking ingredient, a cream, a cheese, or a butter, or a livestock food or a pet food.

In one embodiment, the food or beverage product comprises an alcoholic beverage. In some embodiments, the alcoholic beverage is beer, wine, spirits, or a ready-to-drink (RTD) beverage. In some embodiments, the food or beverage product comprises a low alcohol or non-alcoholic beer or wine.

In one embodiment, the food or beverage product comprises an animal food. In some embodiments, the animal food is a livestock food, a pet food, a kibble, pet biscuit, a pet treat, an animal feed ingredient, a livestock forage, a livestock supplement, or a drench.

In one embodiment, the food or beverage product comprises a bakery good. In some embodiments, the bakery good is a bread, a bar, a ball, a cake, a cookie, or a muffin.

In one embodiment, the food or beverage product comprises a beverage. In some embodiments the beverage is a protein shake, a protein shot, a UHT beverage, a UHT smoothie, a neutral beverage, an acidic beverage, a water, a juice, a milk, a smoothie, a shake, a shot, an alcoholic beverage, a soft drink, a fermented drink, a kombucha, a kefir, a ready-to-drink (RTD) beverage, a coffee beverage, a tea, or a creamer. In some embodiments, the food or beverage product comprises a coffee capsule, an instant coffee, a tea bag, or a coffee creamer.

In one embodiment, the food or beverage product comprises a breakfast food. In some embodiments, the breakfast food is a cereal, oats, a porridge, a spread, a jam, a jelly, or a honey.

In one embodiment, the food or beverage product comprises a confectionary. In some embodiments, the confectionary is a boiled sweet, a candy, a chewing gum, a chocolate bar, a chocolate-coated product, a confectionary bar, a fudge, or a truffle.

In one embodiment, the food or beverage product comprises a dairy product. In one embodiment, the dairy product is a yoghurt. In some embodiments, the yoghurt is a chilled yoghurt, an ambient yoghurt, a stirred yoghurt, a set yoghurt, or a drinking yoghurt. In one embodiment, the dairy product is a milk. In some embodiments, the milk is fresh milk, UHT milk, fortified milk, flavoured milk, or a fermented milk product such as kefir. In one embodiment, the dairy product is a cheese. In some embodiments the cheese is a block cheese, a cream cheese, or a processed cheese. In some embodiments the dairy product is a cream, such as a fresh cream or a UHT cream. In some embodiments, the dairy product is an ice cream.

In one embodiment, the food or beverage product comprises a dessert. In some embodiments, the dessert is an ice cream, a sorbet, a frozen yoghurt, a pudding, a mousse, a jelly, a fruit puree, or a dessert topping.

In some embodiments, the food or beverage product comprises an infant formula, a follow-on formula, a growing-up formula, a paediatric formula, a human milk fortifier, a children's food or drink, a maternal supplement, or a maternal nutritional formulation. In some embodiments, food or beverage product comprises an infant formula powder, a children's milk powder, a wet blend infant formula, a baby food, a baby snack, a baby rice, a baby melting puff, a baby teething rusk, a fruit and/or vegetable puree.

In one embodiment, the food or beverage product is a meal ingredient. In some embodiments the meal ingredient is a condiment, congee (rice porridge), a foam cream, instant noodles, an oil, a pasta, a rice, a salad dressing, or a seasoning.

In one embodiment, the food or beverage product comprises a medical food or beverage. In some embodiments, the medical food or beverage is a meal replacement, a meal replacement shake, a nutritional drink, a soup, a pudding, a nutritional powder, a liquid diet, or a dietary supplement. In some embodiments, the medical food or beverage comprises an encapsulated probiotic and/or an antibiotic.

In one embodiment, the food or beverage product comprises a powder. In some embodiments the powder is a protein powder, a ready-to-mix beverage powder, or a meal replacement powder.

In one embodiment, the food or beverage product comprises a snack. In some embodiments, the snack is a baked good, a cheese lollipop, chips, a bar, a cookie, a dip, dried fruit, nuts, a ball, a fruit leather, a high protein baked good, a yoghurt drop, a grain mix, popcorn, potato chips, a smoothie bar, or a veggie chip.

In some embodiments, the food or beverage product has an extended shelf life when stored at ambient temperature. For example, the food or beverage product may have a shelf life of at least 1 month when stored at ambient temperature, preferably at least 2 months, 3 months, 4 months, 5 months, 6 months, 7 months, 8 months, 9 months, 10 months, 11 months, or 12 months. An extended shelf life of a food or beverage product may be achieved by a number of techniques known in the art, such as ultra-high heat treatment (UHT). In one embodiment, the food or beverage product is a UHT food or beverage product.

In some embodiments, the food or beverage product has a high water activity (aw). It is believed that the particles described herein are particularly useful in high water activity products. In various embodiments, the food or beverage product has a water activity of at least 0.1, preferably at least 0.2, 0.3, 0.4, 0.5, 0.6, 0.7, 0.8, 0.9, or 0.95. In a preferred embodiment, the food or beverage product has a water activity of about 1.0.

In some embodiments, the bioactive agent in the food or beverage product remains active for at least 2 weeks when stored at 30 °C, preferably at least 1 month, 2 months, 3 months, 4 months, 5 months, 6 months, 7 months, 8 months, 9 months, 10 months, 11 months, or 12 months.

In embodiments where the bioactive agent is a microorganism, the degradation rate of the microorganism in the food or beverage product when measured over 12 months at 30 °C may be less than 7 log cfu/g/year, preferably less than 6, 5, 4, 3, 2, or 1 log cfu/g/year.

In some embodiments, when the bioactive agent is a microorganism, the food or beverage product may be formulated to allow the administration of a sufficient amount of the microorganism to establish a population in the gastrointestinal tract of the subject when ingested. The established population may be a transient or permanent population.

In theory one colony forming unit (cfu) should be sufficient to establish a population of microorganisms in a subject, but in actual situations a minimum number of units are required to do so. Therefore, for therapeutic mechanisms that are reliant on a viable, living population of microorganisms, the number of units administered to a subject will affect therapeutic efficacy.

For example, when the bioactive agent is L. rhamnosus HN001, a dosage rate of 6×10⁹ cfu per day is sufficient (but may not be necessary) to establish a population in the gastrointestinal tract of human subjects. Accordingly, in one embodiment, a food or beverage product may be formulated to provide at least about 6×10⁹ cfu L. rhamnosus HN001 per day.

Methods to determine the presence of a population of gut flora, such as L. rhamnosus HN001, in the gastrointestinal tract of a subject are well known in the art. In certain embodiments, presence of a population of microorganisms, such as L. rhamnosus HN001 can be determined directly, for example by analysing one or more samples obtained from a subject, and determining the presence or amount of the microorganism in said sample. In other embodiments, presence of a population of microorganisms can be determined indirectly, for example by observing a health effect, or a decrease in the number of other gut flora in a sample obtained from a subject. Combinations of such methods are also envisaged.

Methods of calculating appropriate dose may depend on the nature of the bioactive agent in the food or beverage product. For example, when the product comprises live microorganisms, the dose may be calculated with reference to the number of live microorganisms present. For example, the dose may be established by reference to the number of colony forming units (cfu) to be administered per day. In examples where the composition comprises one or more derivatives of a microorganism, the dose may be calculated by reference to the amount or concentration of the microorganism derivative present. For example, for a composition comprising a microbial cell lysate, the dose may be calculated by reference to the concentration of the cell lysate present in the composition.

By way of general example, the administration of from about 1×10⁶ cfu to about 1×10¹² cfu of microorganism per kg body weight per day, preferably about 1×10⁶ cfu to about 1×10¹¹ cfu/kg/day, about 1×10⁶ cfu to about 1×10¹⁰ cfu/kg/day, about 1×10⁶ cfu to about 1×10⁹ cfu/kg/day, about 1×10⁶ cfu to about 1×10⁸ cfu/kg/day, about 1×10⁶ cfu to about 5×10⁷ cfu/kg/day, or about 1×10⁶ cfu to about 1×10⁷ cfu/kg/day, is contemplated. Preferably, the administration of from about 5×10⁶ cfu to about 5×10⁸ cfu per kg body weight of microorganism per day, preferably about 5×10⁶ cfu to about 4×10⁸ cfu/kg/day, about 5×10⁶ cfu to about 3×10⁸ cfu/kg/day, about 5×10⁶ cfu to about 2×10⁸ cfu/kg/day, about 5×10⁶ cfu to about 1×10⁸ cfu/kg/day, about 5×10⁶ cfu to about 9×10⁷ cfu/kg/day, about 5×10⁶ cfu to about 8×10⁷ cfu/kg/day, about 5×10⁶ cfu to about 7×10⁷ cfu/kg/day, about 5×10⁶ cfu to about 6×10⁷ cfu/kg/day, about 5×10⁶ cfu to about 5×10⁷ cfu/kg/day, about 5×10⁶ cfu to about 4×10⁷ cfu/kg/day, about 5×10⁶ cfu to about 3×10⁷ cfu/kg/day, about 5×10⁶ cfu to about 2×10⁷ cfu/kg/day, or about 5×10⁶ cfu to about 1×10⁷ cfu/kg/day, is contemplated.

In certain embodiments, periodic dose need not vary with body weight or other characteristics of the subject. In such examples, the administration of from about 1×10⁶ cfu to about 1×10³ cfu of microorganism per day, preferably about 1×10⁶ cfu to about 1×10¹² cfu/day, about 1×10⁶ cfu to about 1×10¹¹ cfu/day, about 1×10⁶ cfu to about 1×10¹⁰ cfu/day, about 1×10⁶ cfu to about 1×10⁹ cfu/day, about 1×10⁶ cfu to about 1×10⁸ cfu/day, about 1×10⁶ cfu to about 5×10⁷ cfu/day, or about 1×10⁶ cfu to about 1×10⁷ cfu/day, is contemplated. Preferably, the administration of from about 5×10⁷ cfu to about 5×10¹⁰ cfu of microorganism per day, preferably about 5×10⁷ cfu to about 4×10¹⁰ cfu/day, about 5×10⁷ cfu to about 3×10¹⁰ cfu/day, about 5×10⁷ cfu to about 2×10¹⁰ cfu/day, about 5×10⁷ cfu to about 1×10¹⁰ cfu/day, about 5×10⁷ cfu to about 9×10⁹ cfu/day, about 5×10⁷ cfu to about 8×10⁹ cfu/day, about 5×10⁷ cfu to about 7×10⁹ cfu/day, about 5×10⁷ cfu to about 6×10⁹ cfu/day, about 5×10⁷ cfu to about 5×10⁹ cfu/day, about 5×10⁷ cfu to about 4×10⁹ cfu/day, about 5×10⁷ cfu to about 3×10⁹ cfu/day, about 5×10⁷ cfu to about 2×10⁹ cfu/day, or about 5×10⁷ cfu to about 1×10⁹ cfu/day, is contemplated.

For example, in one embodiment, an efficacious dose of L. rhamnosus HN001 may be 6×10⁹ cfu per day.

It will be appreciated that the food or beverage product is preferably formulated so as to allow the administration of an efficacious dose of the bioactive agent. The dose of the composition administered, the period of administration, and the general administration regime may differ between subjects depending on such variables as the bioactive agent used, the severity of symptoms of a subject, the type of disorder to be treated, the mode of administration chosen, and the age, sex and/or general health of a subject. Furthermore, as described above the appropriate dose may depend on the nature of the bioactive agent in the food or beverage product and the manner of formulation. For example, when the composition comprises live microorganisms, the dose may be calculated with reference to the number of live microorganisms present. For example, as described herein the examples the dose may be established by reference to the number of colony forming units (cfu) to be administered per day. In examples where the food or beverage product comprises one or more microbial derivatives, the dose may be calculated by reference to the amount or concentration of microbial derivative to be administered per day. For example, for a composition comprising a microbial cell lysate, the dose may be calculated by reference to the concentration of microbial cell lysate present in the composition.

It will be appreciated that preferred food or beverage products are formulated to provide an efficacious dose in a convenient form and amount. In certain embodiments, such as but not limited to those where periodic dose need not vary with body weight or other characteristics of the subject, the products may be formulated for unit dosage. It should be appreciated that administration may include a single daily dose or administration of a number of discrete divided doses as may be appropriate.

### Personal Care Products

In one aspect, the invention provides a personal care product comprising the particle of the first aspect.

The term “personal care product” refers to any product, article, or preparation used for personal care purposes (such as toiletry purposes), including (but not limited to) products for cleaning or grooming oneself, products used in personal hygiene, and products used for beautification. Some examples of personal care products include, but are not limited to, skincare products, haircare products, dental products, deodorants, cosmetics, beauty products, and women's health products.

In one embodiment, the personal care product is a skincare product such as a skin cream, a sunscreen, a haircare product such as a shampoo or a conditioner, a dental product such as a toothpaste or a dentifrice, a deodorant, a cosmetic, a beauty product, or a woman's health product.

In some embodiments, the personal care product is a skincare product. Skincare products are products applied topically to an area of the skin. Some examples of skincare products include moisturisers, lotions, creams, cleansers, and serums. In some embodiments, the skincare product is an eczema cream, a lotion, or a sunscreen.

In some embodiments, the personal care product is a haircare product, such as a shampoo, a conditioner, a hair oil, a hair gel, a hair serum, a hair wax, a hair clay, a pomade, a hair mousse, a dry powder shampoo, or a hair volumizer.

In some embodiments, the personal care product is a dental product, such as a toothpaste, a dentifrice, a dental gel, a dental chew, a mouthwash, or a gargle.

In some embodiments, the personal care product is a deodorant, such as a deodorant stick, a roll-on deodorant, or an antiperspirant.

In some embodiments, the personal care product is a woman's health product.

### Cleaning Products

In one aspect, the invention provides a cleaning product comprising the particle of the first aspect.

In some embodiments, the cleaning product comprises a cleaning spray, a detergent, or a sanitiser.

### Methods of Manufacturing Particles

Various methods of manufacturing the particles of the invention are envisioned. In some embodiments, the method comprises the steps of:
a. contacting the bioactive agent and the matrix to form a mixture, and
b. forming the mixture into particles.

One exemplary method is described below, but various modifications will be immediately apparent to the skilled person without departing from the scope of the invention.

In some embodiments, to prevent contamination, particles are prepared under sanitary and/or aseptic conditions, such as using a Biological Safety Cabinet.

In one embodiment, when the matrix comprises more than one ingredient, the matrix ingredients are combined. The combining can be by any suitable means known in the art, for example by mixing or blending. In some embodiments, the matrix comprise one or more dry ingredients, which are dry-blended together. In some embodiments, one or more dry ingredients (or the dry-blended ingredients) are combined with one or more liquid ingredients. Such liquid ingredients may include ingredients that have been heated to a temperature sufficient to liquify them (for example, one or more lipid ingredients).

In one embodiment, the one or more matrix ingredients are heated to a temperature for a time period sufficient to melt them. The temperature and time required will depend on the composition of the matrix ingredient(s). For example, in some embodiments the matrix ingredient(s) are heated to 65 °C for 30 minutes to melt them.

The heating may be by any suitable means known in the art, for example using a heated water or oil bath.

The heating step may be followed by a tempering step at a lower temperature for a time period sufficient to allow the matrix material to equilibrate at the lower temperature. For example, the ingredients may be held at 45 °C for a minimum of 30 minutes. The tempering step lowers the temperature of the matrix material to prevent heat damage to the bioactive agent, in cases where the bioactive agent is susceptible to heat damage. Many microorganisms such as probiotics are known to be susceptible to heat damage, so in these cases a tempering step would typically be used. For bioactive agents that are not susceptible to heat damage, the tempering step is unnecessary.

The bioactive agent(s) are then added to the (optionally tempered) liquid matrix ingredients and optionally stirred until homogenous. Any additional ingredients may also be added at this stage.

The resultant mixture containing the bioactive agent(s) are then formed into particles and the temperature lowered so that the mixture solidifies. Production of particles may be by any suitable means known in the art, for example by spray chilling or prilling. In some embodiments, the particles are produced by prilling. In other embodiments, the particles are produced by spray chilling. In some embodiments, the mixture is cooled to a temperature below 24 °C, such as 22 °C, 20 °C, 18 °C, 16 °C, 14 °C, 12 °C, 10 °C, 8 °C, 6 °C, 4 °C, 2 °C, or about 0 °C, and useful ranges may be selected between any of these values (for example, from 0 °C to 24 °C, from 0 °C to 10 °C, from 0 °C to 8 °C, from 0 °C to 6 °C, from 0 °C to 4 °C, from 0 °C to 2 °C, from 2 °C to 24 °C, from 2 °C to 10 °C, from 2 °C to 8 °C, from 2 °C to 6 °C, or from 2 °C to 4 °C).

In one embodiment, the mixture is poured into pre-sterilised moulds, and any excess material is removed, for example using a sterile spatula. Moulds are then maintained in sterilised and sealed containers and cooled to allow the mixture to solidify. For example, the moulds may be cooled to 4 °C for 12 h.

The particles are optionally washed and/or filtered by size.

The particles may optionally be coated with one or more coating layers. Coating layers may be applied by techniques known in the art, for example by spray coating.

The particles may optionally be dried, for example by fluidised bed drying, freeze drying, spray drying, or vacuum drying.

The particles may be incorporated into a product (such as a food or beverage product) or they may be stored for future use. Storage may be at ambient temperature and humidity, or alternatively may be under controlled temperature and/or humidity. For example, the particles may be stored chilled (e.g. at about 4 °C) or frozen (e.g. at less than 0 °C), and/or may be stored in a sealed container, optionally a gas-flushed sealed container.

### Methods of Increasing the Stability of a Bioactive Agent

In a fifth aspect the invention provides a method of increasing the stability of a bioactive agent, the method comprising the steps of:
a. embedding the bioactive agent in a biofilm,
b. embedding the biofilm in a matrix, and
c. forming the matrix into particles.

In a sixth aspect, the invention provides a method of increasing the stability of a bioactive agent, the method comprising the steps of:
a. embedding the bioactive agent in a matrix,
b. inducing the bioactive agent to produce a biofilm, and
c. forming the matrix into particles,
   wherein steps b) and c) can be in any order.

In one embodiment, the bioactive agent comprises a first microorganism. In one embodiment, the biofilm is produced by the first microorganism. In some embodiments, the first microorganism is a microorganism as described herein.

In some embodiments, the method further comprises the step of coating the particles in at least one coating layer. In some embodiments, the coating layer is a coating layer as described herein.

In some embodiments, the particle produced in step (c) is a particle according to the first aspect.

In some embodiments, the method further comprises the steps of:
d. combining the particles of step c with a medium having a water activity of at least 0.5, preferably a UHT-treated yoghurt, and
e. incubating the medium for at least 2 weeks.

Without wishing to be bound by theory, it is believed that incubating the particles of step (c) in a medium such as a UHT-treated yoghurt may allow the controlled influx of moisture and/or other compounds present in the medium such as lactose into the particle, stimulating the production of biofilm.

### Methods of Producing a Bioactive Agent Supplemented Food or Beverage Product

In a further aspect the invention provides a method of producing a bioactive agent supplemented food or beverage product, the method comprising the method of the fifth or sixth aspect, and further comprising the steps of:
f. harvesting the particles from the medium of step e, and
g. combining the harvested particles with a food or beverage product, thus obtaining the bioactive agent supplemented food or beverage product.

In another aspect the invention provides a method of producing a bioactive agent supplemented food or beverage product, the method comprising the method of the fifth or sixth aspect, and further comprising the step of combining the particles with a food or beverage product, thus obtaining the bioactive agent supplemented food or beverage product.

In some embodiments, the bioactive agent supplemented food or beverage product is a food or beverage product according to the second aspect.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

The following Examples illustrate the invention.

### EXAMPLES

### 1. Example 1—Coating Matrices for Bioactive Agents

### 1.1 Materials and Methods

Various vegetable oils, individually or in combinations, with and without food ingredient adjuncts, were tested for their ability to provide a protective barrier between a probiotic (Lacticaseibacillus rhamnosus HN001) and a fermented dairy product.

Particles (in the form of beads) were made containing the probiotic ingredient (L. rhamnosus HN001) embedded in a matrix comprising fats and/or combinations of the fats and supporting ingredients. The probiotic ingredient used was a lyophilised commercial sample.

Probiotic matrix beads were prepared by initially melting the lipid ingredients at 65 °C for 30 minutes, and then tempering at 45 °C for a minimum of 30 minutes. The tempering step is to prevent heat damage to the probiotics. To prevent contamination, particles were prepared in a Biological Safety Cabinet. Probiotic bead ingredients were added to tempered lipid ingredients and stirred until homogeneous. Dried ingredients were combined with agitation. The resultant mixture containing the probiotics were poured into pre-sterilised molds, with any excess material removed using a sterile spatula. Molds were maintained in a sterilised and sealed container and cooled to 4 °C for 12 h. Beads were subsequently removed from the mold, and stored in nitrogen-flushed, and heat-sealed bags at −18 °C.

Particles containing probiotics were prepared in the form of beads (about 3-5 mm in diameter as determined by microscopy) using the fats and fat mixtures described in Table 1. The fats formed a continuous layer throughout the bead. All the matrices additionally contained the following adjunct ingredients (typically found in confectionery) as percentages of total solids: 40.7% sucrose, 0.3% sorbitan tristearate (emulsifier 492), 0.5% soy lecithin, 26% milk solids, and 0.12% sodium.

TABLE 1
Composition of fats in coating matrices.

| | Fully | | Fully | |
|---|---|---|---|---|
| | hydrogenated | | hydrogenated | |
| | palm kernel | Palm | coconut | Cocoa |
| Sample ID | stearin | stearin | oil | powder |
| AT01 | 32 | 0 | 0 | 0 |
| AT02 | 0 | 32 | 0 | 0 |
| AT03 | 0 | 0 | 32 | 0 |
| AT04 | 16 | 16 | 0 | 0 |
| AT05 | 16 | 0 | 16 | 0 |
| AT06 | 16 | 0 | 0 | 16 |
| AT07 | 0 | 16 | 16 | 0 |
| AT08 | 0 | 16 | 0 | 16 |
| AT09 | 0 | 0 | 16 | 16 |
| AT10 | 10.7 | 10.7 | 10.7 | 0 |
| AT11 | 10.7 | 10.7 | 0 | 10.7 |
| AT12 | 10.7 | 0 | 10.7 | 10.7 |
| AT13 | 0 | 10.7 | 10.7 | 10.7 |
| AT14 | 8 | 8 | 8 | 8 |
| AT15 | 20 | 4 | 4 | 4 |
| AT16 | 4 | 20 | 4 | 4 |
| AT17 | 4 | 4 | 20 | 4 |

The protected cells were added to a thick-set UHT yoghurt formulated for ambient storage and stored for 12 months at 30 °C to determine their shelf-life. Samples were taken at the commencement of the trial and on a monthly basis, and analysed for cell counts of the HN001 probiotic. Cell degradation rates were determined for each composition. Final samples were taken at 12 months' elapsed time, and a degradation rate was calculated for the probiotics using each coating matrix.

### 1.2 Results

Some of the particles were more structurally robust than others, depending upon composition, however, most survived 12 months' duration of incubation in the yoghurt matrix. Particles comprising high amounts of fully hydrogenated palm kernel stearin were the most robust, followed by hydrogenated palm kernel oil (as described in Example 2), then cocoa butter (as described in Example 2), then hydrogenated coconut oil (data not shown).

A surprising observation was that within the first month of storage, all of the probiotics in the bead particles demonstrated a growth phase, followed by the expected degradation. Without wishing to be bound by theory, it is believed that this may be due to the semi-permeable nature of the beads, which allows some amount of water, lactose, and other nutrients was able to enter into the particle and provide a carbon source for limited growth, followed by a stage of physiological adjustment and stasis.

The analysis of degradation and degradation rates in the bead particles were determined from 1 month into the storage period, as it would be reasonable to assume that the growth and physiological adjustment components are separate processes.

Some probiotic cells were also observed to be released from the bead particles, however the majority of cells remained embedded in the bead.

Degradation rates for each coating matrix are shown in Table 2. Degradation rates are expressed as the log reduction in CFU per gram per year. In other words, degradation rates measured at the 6 and 9 month time periods are extrapolated out to 1 year for clarity in the table.

TABLE 2
Cumulative degradation rates of probiotics
encapsulated in coating matrices.

Degradation rate of probiotics (log cfu/g/y)

| Sample ID | 6 months | 9 months | 12 months |
|---|---|---|---|
| AT01 | 4.1 | 2.4 | 2.9 |
| AT02 | 1.7 | 3.4 | 3.8 |
| AT03 | 1.2 | 1.8 | 0.88 |
| AT04 | 2.5 | 2.9 | 2.9* |
| AT05 | 1.7 | 1.8 | 1.8* |
| AT06 | 2.2 | 4.5 | 6.6 |
| AT07 | 3.9 | 3.6 | 2 |
| AT08 | 4.5 | 8.5 | 5.5 |
| AT09 | 2.6 | 2.6 | 1.9 |
| AT10 | 4.2 | 2.04 | 2.04 |
| AT11 | 2.5 | 4.1 | 6.1 |
| AT12 | 3.0 | 3.03 | 3.0** |
| AT13 | 3.1 | 4.2 | 3.2 |
| AT14 | 2.7 | 3.1 | 6 |
| AT15 | 3.6 | 1.5 | 3.4* |
| AT16 | 2.6 | 1.9 | 4.1 |
| AT17 | 2.6 | 2.6 | 3 |

(*Rate calculated at 9 months elapsed time;
**6 months elapsed time)

### 1.3 Conclusion

This Example shows that the coating matrices can reduce the degradation of probiotic microorganisms in fermented milk products such as yoghurts, even when stored for extended times at ambient or above ambient temperature.

### 2. Example 2—Further Coating Matrices for Bioactive Agents

Supplementary experiments were conducted to test coatings made entirely of fats, the effect of lecithin, and including an uncoated negative control.

### 2.1 Materials and Methods

Probiotic particles were prepared, added to yoghurt, and the probiotic survival monitored over a 12-month period as described in Example 1.

The samples used were:
1. uncoated freeze-dried culture (negative control),
2. 99.5% cocoa butter, 0.5% lecithin,
3. 100% cocoa butter,
4. 99.5% hydrogenated palm kernel oil, 0.5% lecithin,
5. 100% hydrogenated palm kernel oil,
6. 32% hydrogenated palm kernel oil, and
7. 16% hydrogenated palm kernel oil, 16% cocoa powder.

### 2.2 Results

Degradation rates for each coating are shown in Table 3. Degradation rates are expressed as the log reduction in CFU per gram per year. Degradation rates measured at the 6 and 9 month time periods are extrapolated out to 1 year.

TABLE 3
Degradation rates

| | Degradation rate (log cfu/g/y) | | |
|---|---|---|---|
| | 6 | 9 | 12 |
| Coating | months | months | months |
| Uncoated | did not | did not | did not |
| | survive | survive | survive |
| 99.5% cocoa butter, 0.5% lecithin | 2.3 | 2.04 | 4 |
| 100% cocoa butter | 2.5 | 7.3 | 8.9 |
| 99.5% hydrogenated palm 0.5% | 3.1 | 2.4 | 2.5 |
| kernel oil, lecithin | | | |
| 100% hydrogenated palm kernel oil | 2.7 | 2.5 | 3.2 |
| 32% hydrogenated palm kernel oil | 2.9 | 1.8 | 1.8 |
| 16% hydrogenated palm kernel | 2.3 | 1.9 | 1.9 |
| oil, 16% cocoa powder | | | |

The freeze-dried culture (negative control) with no coating did not survive when incubated in yoghurt at 30 °C. The addition of cocoa powder did not appear to contribute to protection of the probiotic against degradation.

Interestingly, cocoa butter demonstrated a low decay up until 6 months, but then degraded at a substantially higher rate after that period.

The hydrogenated palm kernel oil in combination with adjuncts (at 16% and 32% fat), demonstrated further significantly reduced degradation rates over 12 months' storage (1.9 and 1.8 Log cfu/g/y, respectively).

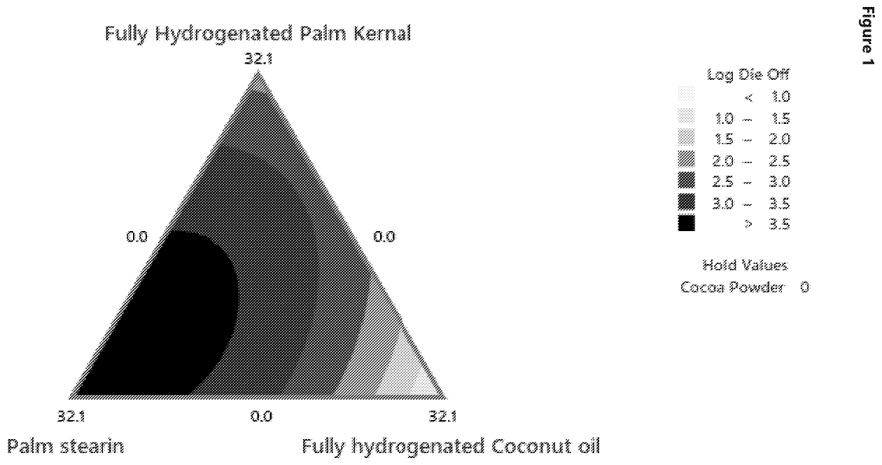

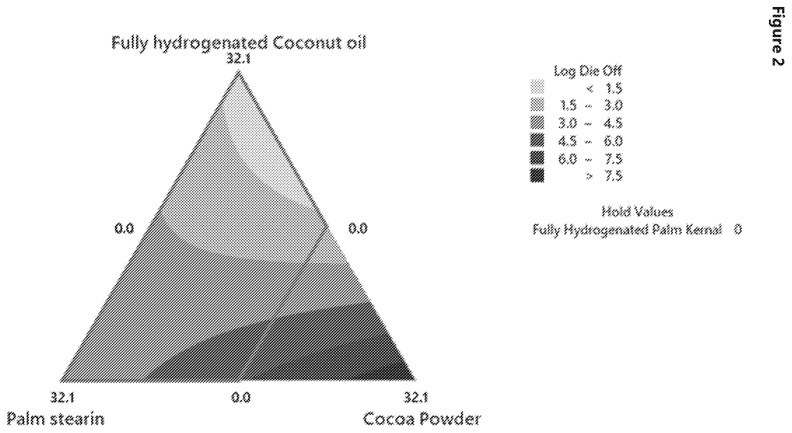

Mixture contour plots were used to further visualise the effects of fats on the stability of the probiotic, individually and in varying combinations. For matrices comprising palm stearin, fully hydrogenated palm kernel oil, and fully hydrogenated coconut oil (individually and in combinations), the lowest die-off was observed when fully hydrogenated coconut oil was added at the highest level (FIG. 1). A similar result was observed for the combination of palm stearin, fully hydrogenated coconut oil, and cocoa powder (FIG. 2).

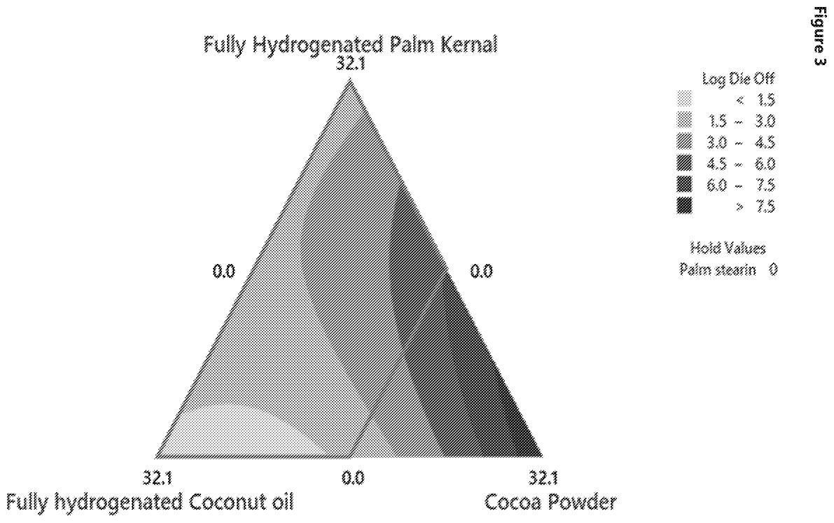

The highest die-off rate was observed when cocoa powder was added at the highest level (FIGS. 2 and 3). This could be removed from the formulation, leaving fully hydrogenated palm kernel oil, fully hydrogenated coconut oil, palm stearin, or combinations thereof as potential protective matrices.

### 2.3 Conclusion

This Example shows that coating matrices comprising 100% fat (i.e. 100% hydrogenated palm kernel oil) can reduce the degradation of probiotic microorganisms in fermented milk products when used alone as a coating matrix. The degradation can be further reduced by including the adjuncts described in Example 1.

This Example also shows that matrices comprising fully hydrogenated coconut oil and/or fully hydrogenated palm kernel oil provide very low die-off rates, palm stearin provides low die-off rates, and cocoa powder is less effective.

### 3. Example 3—Electron Microscopy

### 3.1 Materials and Methods

Bead particles containing the probiotic ingredient (Lacticaseibacillus rhamnosus HN001) and hydrogenated palm kernel oil were made and added to yoghurt as described in Example 1. In this example, beads were removed after nine months for electron microscopy analysis.

The bead was frozen down to −20 °C and fractured using a razor blade. The bead was mounted with the fractured side located upwards, loaded in the scanning electron microscope (SEM TM4000) and cooled down to −30 °C while the chamber vacuum was being pumped down. The sample was then ready to be observed.

### 3.2 Results

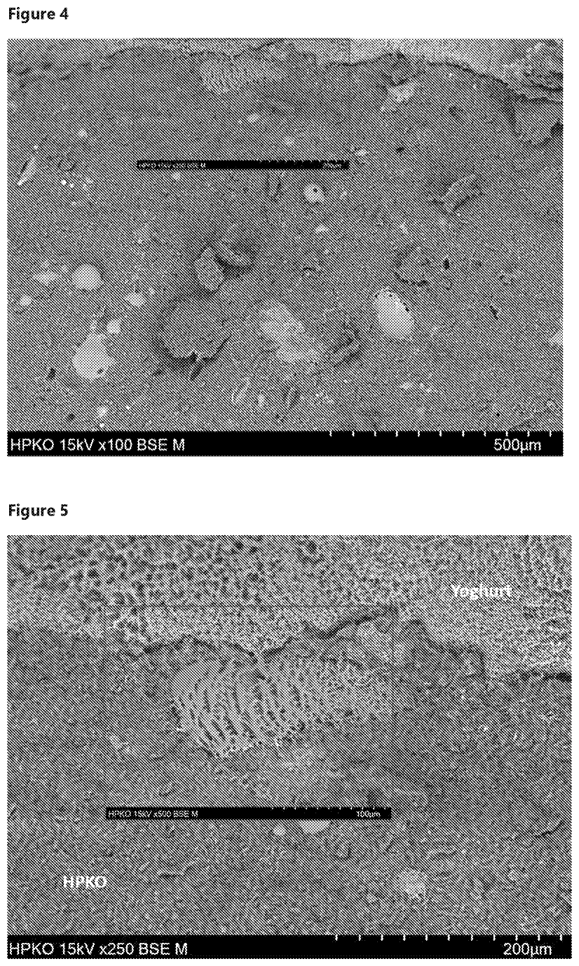

A distinct phase separation between the yoghurt and the fat portion of the composition is visible, as well as distinct clusters of probiotics, which at higher magnification appear to be embedded in a mucoid coating—either exopolysaccharide or a combination with biofilm material (FIG. 4).

FIG. 5 shows a distinct phase separation between the yoghurt and the hydrogenated palm kernel oil (HPKO). The cluster of probiotics in biofilm is demarcated by the red boundary.

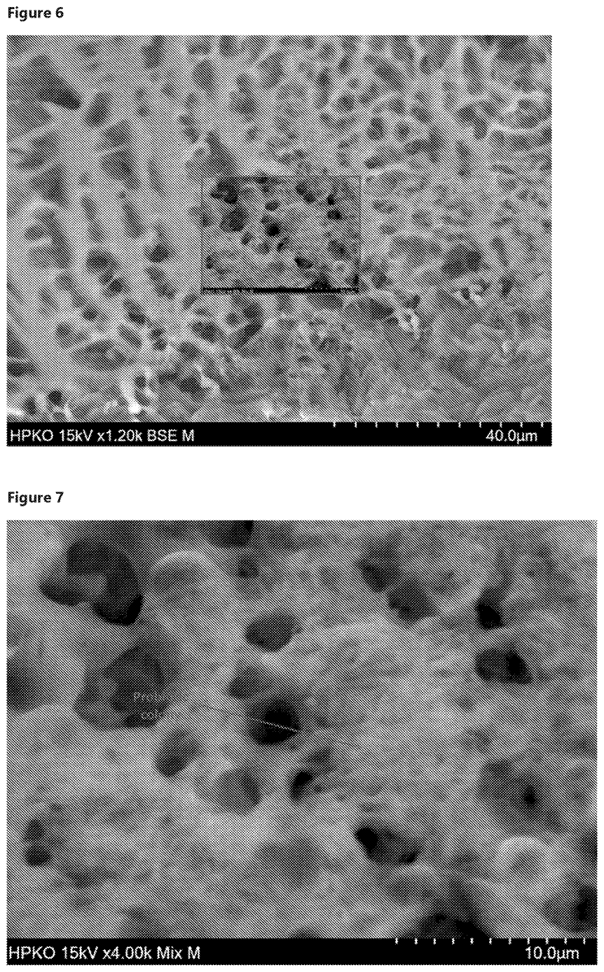

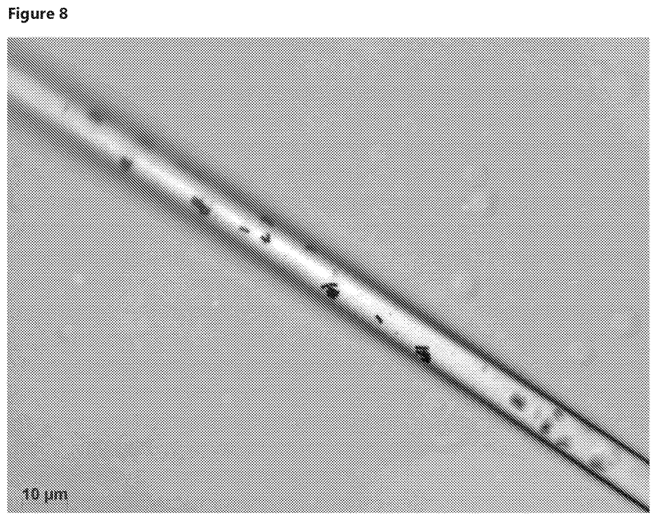

In FIG. 6, the mucoid exopolysaccharide/biofilm network is clearly visible, and in FIG. 7, individual rod-shaped bacterial cells can be seen enmeshed in the exopolysaccharide/biofilm.

### 3.3 Conclusion

This Example shows that probiotic bacteria encapsulated in the coating matrices can develop a mucoid exopolysaccharide or biofilm layer once embedded in the matrix material.

### 4. Example 4—Biofilm Formation

### 4.1 Materials and Methods

Lacticaseibacillus rhamnosus HN001 was demonstrated to produce a biofilm by growth and attachment to a glass wool or activated carbon surface. A culture of HN001 was prepared by incubation in MRSB medium at 37 °C overnight. The culture (1 ml) was then inoculated into a 500 ml flask containing 100 ml of yoghurt. Glass wool (2.5 g) was added to the flask, which was subsequently incubated at 37 °C for 18-24 hours in a rotary shaker incubator. The incubated flasks were then stored at 30 °C, and evaluated at 4, 7, and 28 days for cell number, pH, and EPS or biofilm formation. The same experiment was undertaken with 1 g of 0.6-1.1 mm washed granular activated carbon to the flasks.

Samples were removed at commencement of storage, and at 7 and 28 days for enumeration of viable probiotic cells. The glass wool (or activated carbon) was removed and rinsed 3 times through a mini sieve with phosphate buffered water. The rinsed material was placed into a 250 mL Schott bottle containing 20 g of sterile glass beads. Salted buffered peptone water (9 ml) was added and the beads and culture were agitated vigorously for 10 mins to detach the cells from the glass wool or charcoal.

Serial dilutions using 9 ml Salted Buffered Peptone water were undertaken, and plated out onto MRSA agar, followed by incubation at 37 °C for 48 hour, prior to counting of the cells. Cell counts were also undertaken on the residual yoghurt (planktonic or free cells).

### 4.2 Results

Table 4 demonstrates that a significant number of viable HN001 cells remained attached to the glass wool or charcoal surfaces over the 28-day period of shelf-life, maintaining a relatively higher level of survival than free cells in the yoghurt.

TABLE 4
Comparison of cell counts and pH of cells attached to glass or charcoal
surfaces, with cells remaining unattached (planktonic cells).

| | | Time (days) | | |
|---|---|---|---|---|
| | Sample | 0 | 7 | 28 |
| HN001 (log | Glass wool | 8.76 | 8.06 | 8.08 |
| cfu/g) | (biofilm) | | | |
| | Charcoal | 7.26 | 8.47 | 8.93 |
| | (biofilm) | | | |
| | Glass wool | N/A | 7.19 | 6.07 |
| | (Planktonic) | | | |
| | Charcoal | N/A | 7.07 | 7.06 |
| | (Planktonic) | | | |
| pH | Yoghurt with | 4.22 | 3.85 | 3.49 |
| | glass wool | | | |
| | Yoghurt with | 4.22 | 4.02 | 3.64 |
| | charcoal | | | |

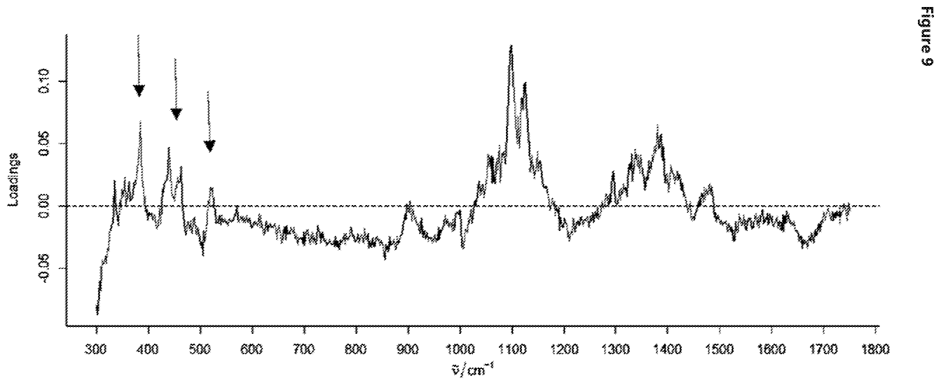

Samples attached to glass wool were studied at 100× magnification using light microscopy under oil-immersion, after staining with crystal violet (FIG. 8). Clusters of HN001 cells were visible, attached to the surface of the glass wool strand. These attached clusters provide evidence of the ability of HN001 to form a biofilm, sustaining the viability of the cells in yoghurt.

### 4.3 Conclusion

This Example shows that Lacticaseibacillus rhamnosus HN001 is capable of forming a biofilm, and that doing so can improve the viability of the cells when stored in yoghurt for extended periods.

### 5. Example 5—Identification of Biofilm Exopolysaccharide Components Using RAMAN Spectroscopy

Exopolysaccharides (EPSs) constitute the major component of extracellular biofilm matrices, and have the effect of anchoring microorganisms to surfaces, a process which initiates biofilm formation.

The signature sugars of the EPS produced by Lacticaseibacillus rhamnosus HN001 EPS are glucose, galactose and rhamnose with trace amounts of galactosamine, glucosamine, mannose and fucose. Because residual glucose and galactose is likely to be found in yoghurt, identification of rhamnose, galactosamine, glucosamine, mannose and fucose provides strong evidence of the presence of biofilm associated with HN001.

### 5.1 Materials and Methods

Beads containing L. rhamnosus HN001 were prepared according to Example 1.

A bead from sample AT03 (see Example 1) was removed from the yoghurt and gently rolled on paper towel to remove excess yoghurt. Sections of beads were cut to about 2 mm thick. Sections were placed on a slide and covered with a cover slip; double sided tape was used to hold the coverslip down and act as a ~2 mm spacer. The samples were maintained at a temperature of 4 °C throughout measurements. Raman maps of 50×10 steps, 75×25 μm were collected. Each spectrum was collected using a 1 s integration time, 532 nm excitation wavelength, 100×oil immersion objective (NA 1.30), 50 μm pinhole. At least ten maps per bead were collected at each time point: 0, 15, 30, 90/120, 218 days.

As a control, a sample of L. rhamnosus HN001 was grown using ambient yoghurt as the medium and a zinc selenide surface to induce biofilm production.

Raman data files were converted to text files for subsequent analysis in R (version 4.1.1) and R Studio (version 1.4.1717). Spectra were pre-processed using cosmic ray rejection, baselining and minimum-maximum normalisation. Raman spectral maps were analysed using principal components analysis, multivariate curve resolution and comparison to reference materials.

### 5.2 Results

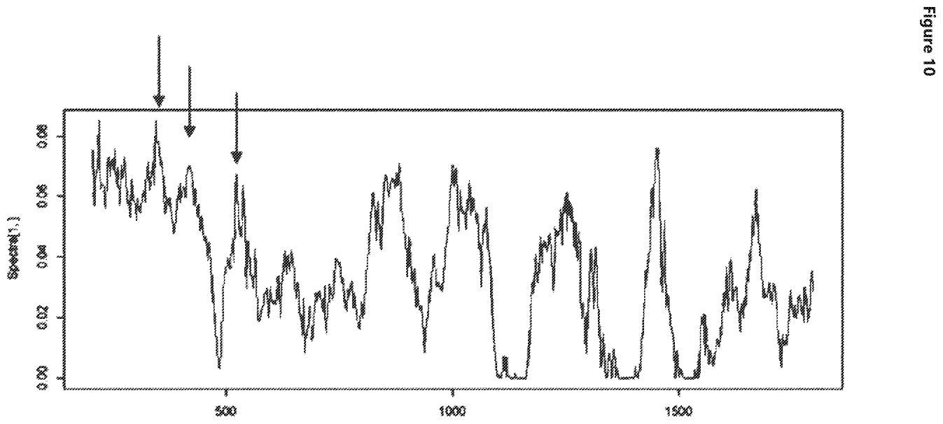

A principal components analysis of Raman spectroscopy data of L. rhamnosus HN001 grown using ambient yoghurt as the medium and a zinc selenide surface to induce biofilm production revealed three distinct peaks in the 350-600 cm⁻¹ spectral region related to the presence of rhamnose (FIG. 9). These may indicate the presence of EPS components.

Raman spectroscopy of sample AT03 bead structures incorporating L. rhamnosus HN001 revealed similar peaks (FIG. 10). Again, three EPS-related peaks appear at 360, 430, and 530 cm⁻¹.

### 5.3 Conclusion

This Example shows that Raman spectroscopy can be used to detect the presence of EPS in probiotic-containing particles.

### 6. Example 6—Identification of Biofilm Exopolysaccharides by Lectin Staining

### 6.1 Materials and Methods

Beads containing L. rhamnosus HN001 were prepared according to Example 1.

A bead was removed from the yoghurt and gently rolled on paper towel to remove excess yoghurt. Sections of beads were cut to about 2 mm thick. The cut sections were stained with 5 μl of 0.1 mg/ml fluorescein-tagged lectin stain, 1 μM DAPI and 0.2% fast green. Lectins stains were concanavalin A, wheat germ agglutinin, and Ulex europaeus agglutinin. The samples were allowed to stain for at least 15 minutes before imaging using an inverted Zeiss LSM800 confocal fluorescence microscope. A 407 nm laser was used to excite DAPI, a 488 nm laser was used to excite fluorescein tagged lectins, and a 561 nm excitation was used for fast green.

### 6.2 Results

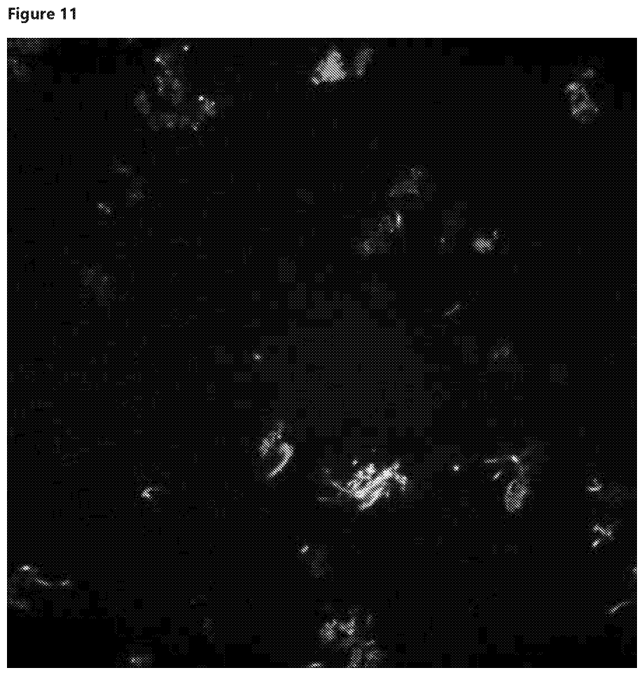

FIGS. 11-13 show fluorescence microscopy of a bead stained with different lectins. The figures are of one field of view recorded using a 63× oil immersion objective. One bead was divided so as to stain different sections with the different stain mixtures.

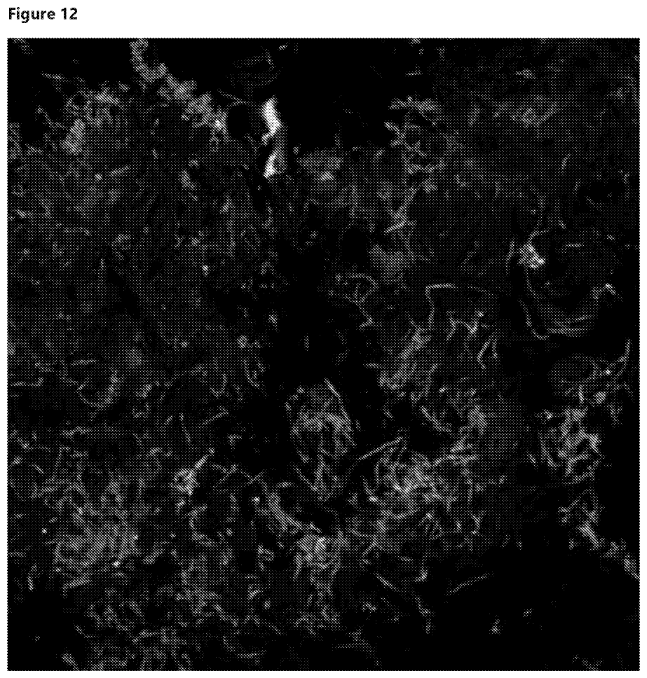

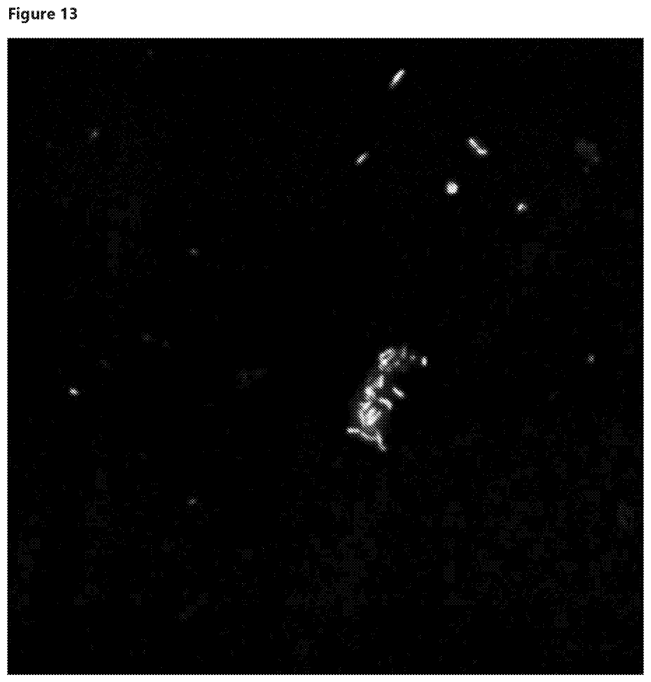

Lectin staining indicated the presence of sugars consistent with the structure of L. rhamnosus HN001 EPS (glucose, galactose, rhamnose, galactosamine, glucosamine, mannose and fucose). The presence of mannose (FIG. 11), N-acetylglucosamine (FIG. 12) and fucose (FIG. 13) provided strong evidence of the presence of biofilm associated with HN001 in the bead particles.

### 6.3 Conclusion

This Example shows that lectin staining and fluorescence microscopy can be used to detect the presence of biofilm in the particles.

### 7. Example 7—Further Coating Matrices

### 7.1 Materials and Methods

Beads comprising Lacticaseibacillus rhamnosus HN001 were prepared as described in Example 1, using the matrix formulations shown in Table 5.

TABLE 5
Matrix compositions (% w/w).

| Sample | PS | HCO | CP | HPKS | HPKO | CB | Suc | 492 | Lec | SMP | NaCl | Lac |
|---|---|---|---|---|---|---|---|---|---|---|---|---|
| AT03 | | 32 | | | | | 41 | 0.3 | 0.5 | 26 | 0.12 | |
| AT03-1 | | 74 | | | | | | | | 26 | | |
| AT03-2 | | 59 | | | | | 41 | | | | | |
| AT07 | 16 | 16 | | | | | 41 | 0.3 | 0.5 | 26 | 0.12 | |
| AT07-1 | 50 | 50 | | | | | | | | | | |
| AT07-2 | 37 | 37 | | | | | | | | 26 | | |
| AT07-3 | 29.5 | 29.5 | | | | | 41 | | | | | |
| AT17 | 4 | 20 | 4 | 4 | | | 41 | 0.3 | 0.5 | 26 | 0.12 | |
| AT17-1 | 19 | 62 | | 19 | | | | | | | | |
| AT17-2 | 14 | 46 | | 14 | | | | | | 26 | | |
| AT17-3 | 11 | 11 | | 37 | | | 41 | | | | | |
| AT21 | | | | | | 99.5 | | | 0.5 | | | |
| AT21-1 | | | | | | 73.5 | | | 0.5 | 26 | | |
| AT24 | | | | | 100 | | | | | | | |
| ATX-1 | | | | | | 86.5 | | | | | | 13.5 |
| ATX-2 | | 86.5 | | | | | | | | | | 13.5 |

PS, palm stearin; HCO, fully hydrogenated coconut oil; CP, cocoa powder; HPKS, fully hydrogenated palm kernel stearin; HPKO, hydrogenated palm kernel oil; CB, cocoa butter; Suc, sucrose; 492, sorbitan tristearate (emulsifier 492); Lec, soy lecithin; SMP, milk solids (medium heat skim milk powder); Lac, lactose.
Beads were added to a less viscous UHT yoghurt (rather than the thick-set yoghurt used in Example 1) and were incubated at 25 or 30 °C for 12 months, with samples removed at various timepoints for cell count analysis.

### 7.2 Results

### Incubation at 30 °C

Cell counts of L. rhamnosus HN001 after incubation at 30 °C are shown in Table 6.

TABLE 6
Cell counts of L. rhamnosus HN001 (log
CFU/g particle) after incubation at 30 °C.

| | Incubation time (months) | | | | | | | |
|---|---|---|---|---|---|---|---|---|
| Sample | 0 | 0.5 | 1 | 2 | 4 | 6 | 9 | 12 |
| AT03 | 9.08 | 10.37 | 9.65 | 9.26 | 9.10 | 8.71 | * | * |
| AT03-1 | 8.99 | 9.62 | 9.37 | 8.01 | 8.53 | 4.46 | * | * |
| AT03-2 | 8.97 | 9.64 | 9.44 | 9.09 | 8.24 | 7.51 | 4.23 | * |
| AT07 | 8.74 | 9.86 | 9.78 | 9.30 | 8.07 | 7.46 | 2.40 | 2.40 |
| AT07-1 | 8.74 | 9.05 | 9.80 | 8.59 | 7.05 | 6.46 | * | * |
| AT07-2 | 8.47 | 9.17 | 9.83 | 8.92 | 7.57 | 6.90 | * | * |
| AT07-3 | 8.87 | 9.65 | 9.89 | 9.17 | 9.22 | 8.57 | 6.90 | 2.48 |
| AT17 | 9.09 | 10.60 | 9.76 | 9.40 | 9.18 | 8.67 | 2.24 | 2.24 |
| AT17-1 | 8.90 | 8.57 | 8.92 | 8.07 | 7.12 | 6.48 | 2.40 | 2.40 |
| AT17-2 | 9.10 | 9.80 | 9.53 | 8.58 | 7.13 | 6.15 | 4.40 | 2.48 |
| AT17-3 | 8.97 | 9.20 | 9.45 | 8.75 | 8.47 | 8.07 | 4.80 | 4.91 |
| AT21 | 8.96 | 9.21 | 9.67 | 9.32 | 9.29 | 8.74 | 7.81 | 7.63 |
| AT21-1 | 9.08 | 10.16 | 9.95 | 9.65 | 9.52 | 8.02 | 6.19 | 6.49 |
| AT24 | 8.98 | 8.74 | 9.01 | 8.75 | 8.42 | 6.96 | 6.45 | 4.26 |
| ATX-1 | 9.08 | 9.53 | 9.39 | 9.22 | 9.25 | 8.61 | 8.11 | 8.10 |
| ATX-2 | 8.90 | 8.30 | 9.30 | 8.10 | 8.95 | 7.63 | 6.91 | 5.52 |

* less than 100 colonies per plate, below the level of accurate determination.

### Incubation at 25 °C

Cell counts of L. rhamnosus HN001 after incubation at 25 °C are shown in Table 7.

TABLE 7
Cell counts of L. rhamnosus HN001 (log
CFU/g particle) after incubation at 25 °C.

| | Incubation time (months) | | | | | | | |
|---|---|---|---|---|---|---|---|---|
| Sample | 0 | 0.5 | 1 | 2 | 4 | 6 | 9 | 12 |
| AT03 | 8.95 | 10.11 | 10.16 | 9.32 | 9.35 | 9.13 | 7.33 | 7.27 |
| AT03-1 | 9.04 | 8.96 | 8.72 | 8.71 | 7.31 | 5.80 | 5.77 | 3.44 |
| AT03-2 | 9.00 | 8.79 | 9.56 | 8.98 | 7.23 | 8.87 | 6.62 | 6.57 |
| AT07 | 8.97 | 9.88 | 9.78 | 9.43 | 9.05 | 8.15 | 8.10 | 6.52 |
| AT07-1 | 9.11 | 7.93 | 8.52 | 8.53 | 8.98 | 6.36 | 6.98 | 6.75 |
| AT07-2 | 8.67 | 8.70 | 9.04 | 8.90 | 6.16 | 7.62 | 6.36 | 6.61 |
| AT07-3 | 8.87 | 8.83 | 9.54 | 9.06 | 8.98 | 8.43 | 7.36 | 6.81 |
| AT17 | 8.92 | 10.25 | 10.41 | 9.45 | 9.45 | 9.47 | 8.60 | 7.25 |
| AT17-1 | 8.93 | 8.88 | 8.95 | 8.90 | 7.09 | 7.59 | 6.52 | 5.11 |
| AT17-2 | 9.03 | 9.53 | 9.50 | 9.43 | 7.98 | 7.49 | 6.71 | 5.91 |
| AT17-3 | 9.09 | 9.01 | 9.55 | 8.93 | 9.04 | 7.09 | 6.39 | 6.41 |
| AT21 | 8.57 | 9.25 | 9.41 | 9.05 | 7.74 | 8.39 | 8.69 | 5.84 |
| AT21-1 | 8.98 | 9.67 | 9.70 | 9.13 | 7.88 | 8.13 | 8.66 | 4.56 |
| AT24 | 9.01 | 8.55 | 8.59 | 8.82 | 6.05 | 7.45 | 7.04 | 6.62 |
| ATX-1 | 8.89 | 9.53 | 9.67 | 9.03 | 9.58 | 9.49 | 9.16 | 8.35 |
| ATX-2 | 9.01 | 8.18 | 8.12 | 7.87 | 8.35 | 8.02 | 7.18 | 6.45 |

### 7.3 Conclusion

This Example shows that a variety of coating matrices can reduce the degradation of probiotic microorganisms in fermented milk products, with some formulations resulting in significant stability.

### 8. Example 8—Biofilm Formation by Lectin Staining

### 8.1 Materials and Methods

Beads containing L. rhamnosus HN001 were prepared according to Example 1 using the matrix formulations described in Table 8, with 2.5% HN001

TABLE 8
Matrix composition.

| Formulation | Matrix composition (w/w) |
|---|---|
| AT07 | 16% hydrogenated coconut oil, 16% palm stearin, 41% sucrose, 0.3% |
| | sorbitan tri-stearate, 0.5% soy lecithin, 26% milk solids, 0.12% sodium |
| | chloride |
| AT24 | 100% hydrogenated palm kernel oil |
| AT17-1 | 62% hydrogenated coconut oil, 19% palm stearin, 19% hydrogenated |
| | palm kernel stearin |
| ATX-1 | 86.5% cocoa butter, 13.5% lactose |

Beads were incubated in a thick-set UHT yoghurt at 30 °C as described in Example 1, and samples were removed at various timepoints for analysis.

Probiotic-containing beads were removed from the yoghurt and excess yoghurt was removed. Timepoint zero particles were used prior to loading into yoghurt.

Lectins bound to fluorophores were used to dye the fat-based beads for confocal fluorescence microscopy. Fluorescently labelled lectins have been used previously to study extracellular polymeric substance in biofilms, as lectins bind to sugar residues present in the polysaccharides of extracellular polymeric substance.

Three lectins were selected for use on the beads: Wheat germ agglutinin (WGA), concanavalin A (ConA), and Ulex europaeus agglutinin I (UEA-I-I). WGA is specific for N-acetylglucosamine, ConA is specific for α-mannose and α-glucose, and UEA-I-I is specific for α-fucose. These were used in combination with DAPI to stain cells, Nile red to stain fat, or fast green to stain protein.

The beads were cut to expose the inner surface and mixtures of fluorescence dyes were applied as shown in Table 9. At most, 10 μl of the fluorescent dye mix was applied to the exposed surface of the bead, and the bead was allowed to be in contact with the dye for at least 15 and no more than 30 minutes prior to imaging.

TABLE 9
Dye mixtures.

| Dye mix | Dye 1 | Dye 2 | Dye 3 |
|---|---|---|---|
| 1 | DAPI 0.1 | WGA-640 0.1 mg/ml | Nile red 0.5% w/v |
| | mg/ml | | |
| 2 | DAPI 0.1 | ConA-640 0.1 mg/ml | Nile red 0.5% w/v |
| | mg/ml | | |
| 3 | DAPI 0.1 | UEA-I-I-640 0.1 mg/ml | Nile red 0.5% w/v |
| | mg/ml | | |
| 4 | DAPI 0.3 | Fast green 0.2% w/v | Nile red 0.5% w/v |
| | mg/ml | | |
| 5 | DAPI 0.1 | WGA-FI 0.1 mg/ml | Fast green 0.2% w/v |
| | mg/ml | | |
| 6 | DAPI 0.1 | ConA-FI 0.1 mg/ml | Fast green 0.2% w/v |
| | mg/ml | | |
| 7 | DAPI 0.1 | UEA-I-I-Fl 0.1 mg/ml | Fast green 0.2% w/v |
| | mg/ml | | |

An inverted Zeiss LSM 800 microscope with 63× oil immersion objective was used to collect confocal fluorescence images from the dyed beads. The excitation wavelengths and emission collection ranges are listed in table 10.

TABLE 10
Excitation and emission wavelengths.

| Dye | Excitation (nm) | Emission collection range (nm) |
|---|---|---|
| DAPI | 405 | 400-595 |
| WGA-640R | 640 | 656-700 |
| ConA-640R | 640 | 656-700 |
| UEA-I-I Dylight 649 | 640 | 656-700 |
| Fast green | 640 | 656-700 |
| Nile red | 561 | 570-700 |
| WGA-Fluorescein | 488 | 410-530 |
| ConA-Fluorescein | 488 | 410-530 |
| UEA-I-I-Fluorescein | 488 | 410-530 |

### 8.2 Results

### AT07

At time zero, differential fluorescence of WGA was observed, indicating that N-acetylglucosamine is present in the initial composition. The WGA binding was largely around the edges of protein bodies. This binding could be a consequence of the inclusion of milk solids in the particle as bovine milk contains N-acetylglucosamine (Li et al. (2023) Quantification of cow milk in adulterated goat milk by HPLC-MS/MS using N-acetylglucosamine as a reliable biomarker of cow milk. Journal of Food Composition and Analysis, 105583).

After three months of incubation there were definite regions of concentrated WGA fluorescence, and these regions were no longer on the edges of protein bodies but instead within cell masses.

At time zero, areas of dense ConA fluorescence were observed, and these areas did not coincide with regions of cell density. This is also likely a consequence of the milk solids included in this matrix formulation.

After three months of incubation there was an increase in the area of appreciable ConA fluorescence, and these areas were associated with regions of higher cell density.

No data was collected from time zero using the UEA-I-I lectin dye. After three months of incubation there were definite regions of high and low UEA-I-I fluorescence, and the regions of high UEA-I-I fluorescence were largely associated with cell-dense areas. This increase in areas of lectin fluorescence about cell masses after incubation accords with the formation of biofilm.

### AT24

No data was collected from time zero using the ConA or UEA-I-I dyes.

At time zero, WGA dyed the cells. After three- and six-months incubation, WGA fluorescence was observed around the cells; this was most obvious after six months. After six months, ConA fluorescence was apparent in cell-dense regions. For UEA-I-I fluorescence there was a small amount of aggregation of UEA-I-I at three and six months, however, it did not seem intimately associated with the cell locations. These observations are consistent with biofilm formation.

### AT17-1

The lectin dyes used to stain MVP9 had a fluorophore with an excitation maximum at 642 or 649 nm, allowing concurrent staining with Nile red, which dyes fats. There was one exception, for the one-month time point—a UEA-I-I dye with a fluorescein fluorophore was used, which has an excitation maximum of 495 nm. When using this dye, Nile red was not used due to interference between the dyes, and instead fast green was added.

At time zero, small areas of bacteria were present within the fat matrix, and WGA dyed the cells in these regions. After one month, more extensive fluorescence from WGA around the cells regions was apparent, indicating the presence of N-acetylglucosamine that extended beyond the immediate area of the cells, consistent with the formation of a biofilm with extracellular polymeric substance containing N-acetylglucosamine.

At time zero there was no differential fluorescence observed when using ConA, but areas of fluorescence were visible where the dye had pooled in non-fat regions. Within cell regions, there were no areas of weak and strong ConA fluorescence. There were regions of ConA fluorescence that did not appear to be associated with cells. Hence, the dye was pooling in non-fat regions rather than specifically dyeing mannose or glucose. After one month, differential fluorescence of ConA was observed, i.e., fluorescence was not just due to pooling in non-fat regions. As with WGA, areas of uneven and extensive fluorescence about cells was observed, consistent with the excretion of extracellular polymeric substances and the formation of biofilm. Not all cell regions were dyed with ConA; at this time point there were cell areas where ConA had just pooled. Further, there were regions of intense aggregation of ConA at one month in regions where cells surrounded the edge and ConA pooled inside but also aggregated within the non-fat region.

At time zero, UEA-I-I dyed cells in the beads in a similar manner to WGA. These cells were largely confined to clusters of cells within the fat matrix. At one month, cells had colonised the outer area of non-fat regions in the particle, and fluorescence due to UEA-I-I fluorescence was observed beyond the cells, consistent with biofilm formation.

### ATX-1

Only three-month samples were analysed.

After three months, there were regions of significant WGA fluorescence associated with areas of cell density. There were also regions of significant ConA fluorescence, also associated with cells, and small regions of UEA-I-I fluorescence. All of these observations are consistent with biofilm formation.

### 8.3 Conclusion

This Example shows that biofilm formation can be detected by fluorescence microscopy using fluorescein-linked lectins, and that various matrix compositions are conducive to forming biofilm.

### 9. Example 9—Stability in Various Products

### 9.1 Materials and Methods

Probiotic particles comprising 0.25% Lacticaseibacillus rhamnosus HN001 were prepared using four matrix formulations as described in Table 11.

TABLE 11
Matrix composition.

| Formulation | Matrix composition (w/w) |
|---|---|
| AT07-3 | 29.5% palm stearin, 29.5% hydrogenated coconut oil, 41% sucrose |
| AT21 | 99.5% cocoa butter, 0.5% soy lecithin |
| AT24 | 100% hydrogenated palm kernel oil |
| ATX-2 | 86.5% hydrogenated coconut oil, 13.5% lactose |

Dry ingredients (sugar, lactose) were weighted into stomacher bags, sealed with a bag sealer, and stored at room temperature until needed. Lipid ingredients (fats and lecithin) were weighted into sterile bottles and stored at 3-4 °C until needed. 0.25 g/100 g lipid of the L. rhamnosus HN001 were weighed into sterile containers and stored at 3-4 °C until needed. Silicon molds were autoclaved.

Particles were prepared using the following method:
1. Lipid ingredients were melted at 60-70 °C in a water bath.
2. The lipid ingredients were transferred to a water bath at 45 °C, making sure that the lipids and fats cooled to 45 °C before addition of probiotics (minimum of 30 min).
3. Probiotics (0.25 g) was added to the melted lipid, stirring using a sterilised spoon, making sure that it was homogeneous.
4. The remaining dry ingredients were added slowly, stirring in with each addition.
5. Agitation was maintained throughout to ensure that all ingredients were combined, resulting in a smooth appearance.
6. The contents were poured into sterilised silicon molds.
7. Using a spatula, the mixture was smoothed into the mold cavities, and the excess scraped off.
8. The molds were placed in pre-sanitised containers and stored at 4 °C for 24 hours.

The particles produced had a diameter of approximately 5 mm and a weight of approximately 0.05 g each.

Five products were evaluated, as described in Table 12. Viscosity of the products was determined using a Brookfield viscometer.

TABLE 12
Products evaluated.

| Product type | Product name | Parameters |
|---|---|---|
| Acidic beverage | Keri ® orange juice | pH: 3.71 |
| | | Viscosity: 3.16 cP |
| Neutral beverage | Fonterra ® sports shake | pH: 6.74 |
| | | Viscosity: 26.8 cP |
| Food product | Dolmio ® cheese sauce | pH: 3.88 |
| | | Viscosity: 2130 cP |
| Food product | Bega ® cream cheese spread | pH: 5.49 |
| | | Viscosity: 1557 cP |
| High water activity product | Vitasoy ® prebiotic oat milk | pH: 6.49 |
| | | Viscosity: 4.68 cP |

200 ml samples of each product were used, and three particles were added to each sample using sterile forceps, immersing the particles in the top 10-20 ml. The samples were then incubated at 30 °C.

At each sampling timepoint, three particles were recovered and placed into a bell tube. 15 ml of MRSB medium, pre-warmed to 45 °C, was added and samples were melted in a 45 °C waterbath for 5 minutes. Samples were removed from the water bath and gently shaken/vortexed to mix.

Dilutions were prepared in duplicate. Appropriate sample dilutions (50 μl) were placed onto a pre-poured agar plate (‘drop plate method’). Three dilutions, namely 10⁻⁶, 10⁻⁷, and 10⁻⁸, were required to obtain a countable range.

The culture plates were incubated at 37 °C for 2-3 days under anaerobic conditions, and the number of colonies counted to determine the CFU/ml in the original sample.

### 9.2 Results

The counts of viable L. rhamnosus HN001 at initial set up, and after 1, 2, 3, and 4 months incubation, are shown in Tables 13-17. The 3 and 4-month timepoints have yet to be tested for the cream cheese spread.

TABLE 13
Shelf life in orange juice (Log₁₀ CFU/ml).

Time (months)

| Formulation | 0 | 1 | 2 | 3 | 4 |
|---|---|---|---|---|---|
| AT07-3 | 8.41 | 8.49 | 7.93 | 6.33 | 3.13 |
| AT21 | 8.54 | 8.27 | 5.78 | <10⁻³ | 2.56 |
| AT24 | 8.87 | 7.93 | 6.40 | 4.30 | <10⁻² |
| ATX-2 | 9.23 | 8.52 | 7.13 | <10⁻³ | 2.23 |

TABLE 14
Shelf life in sports shake (Log₁₀ CFU/ml).

| | Time (months) | | | | | |
|---|---|---|---|---|---|---|
| Formulation | 0 | 1 | 2 | 3 | 4 | 6 |
| AT07-3 | 8.41 | 9.12 | 8.36 | 7.03 | 7.94 | 7.27 |
| AT21 | 8.54 | 8.75 | 7.08 | 6.28 | 6.85 | 6.76 |
| AT24 | 8.87 | 8.48 | 6.48 | 5.81 | 6.47 | 6.17 |
| ATX-2 | 9.23 | 9.35 | 8.37 | 8.07 | 8.00 | 7.57 |

TABLE 15
Shelf life in cheese sauce (Log₁₀ CFU/ml).

| | Time (months) | | | | | |
|---|---|---|---|---|---|---|
| Formulation | 0 | 1 | 2 | 3 | 4 | 6 |
| AT07-3 | 8.41 | 9.35 | 9.13 | 8.72 | 9.18 | 8.54 |
| AT21 | 8.54 | 9.34 | 8.80 | 7.79 | 8.96 | 8.70 |
| AT24 | 8.87 | 9.23 | 8.41 | 6.56 | 8.18 | 6.68 |
| ATX-2 | 9.23 | 9.17 | 7.77 | 5.53 | 7.12 | 7.22 |

TABLE 16
Shelf life in cream cheese spread (Log₁₀ CFU/ml).

Time (months)

| Formulation | 0 | 1 | 2 | 4 |
|---|---|---|---|---|
| AT07-3 | 9.79 | 9.85 | 9.14 | 9.16 |
| AT21 | 9.63 | 9.20 | 8.66 | 8.16 |
| AT24 | 8.60 | 8.51 | 8.37 | 7.81 |
| ATX-2 | 8.52 | 9.30 | 8.99 | 8.93 |

TABLE 17
Shelf life in prebiotic oat milk (Log₁₀ CFU/ml).

| | Time (months) | | | | | |
|---|---|---|---|---|---|---|
| Formulation | 0 | 1 | 2 | 3 | 4 | 6 |
| AT07-3 | 8.41 | 8.64 | 6.19 | 5.53 | <10⁻² | <10⁻² |
| AT21 | 8.54 | 7.62 | 7.47 | <10⁻³ | <10⁻² | <10⁻² |
| AT24 | 8.87 | 7.58 | 5.78 | <10⁻³ | 2.12 | <10⁻² |
| ATX-2 | 9.23 | 7.83 | 5.60 | 7.65 | 6.33 | 5.03 |

### 9.3 Conclusion

This Example shows that coating matrices can reduce the degradation of probiotic microorganisms in a wide variety of products, even when stored for extended times at ambient or above ambient temperature.

### 10. Example 10—Bacterial Strains

### 10.1 Materials and Methods

Two sets of particles were prepared as described in Example 9, the first comprising Lacticaseibacillus rhamnosus HN001 and the second comprising Lacticaseibacillus paracasei subsp. paracasei IM514.

The matrix composition contained 86.5% w/w hydrogenated coconut oil (HCO) and 13.5% w/w lactose (formulation ATX-2). Before adding the bacteria to the melted lipid ingredients, a further cooling step of one hour at 20 °C was applied.

Particles were added into a thick-set UHT yoghurt formulated for ambient storage, and stored at 30 °C. Cell counts of the respective bacterial strains were taken periodically.

### 10.2 Results

Cell counts of the bacterial strains are shown in Table 18.

TABLE 18
Shelf life in UHT yoghurt (Log₁₀ CFU/ml).

| | Time (months) | | | | |
|---|---|---|---|---|---|
| Bacteria | 0 | 0.5 | 1 | 2 | 4 |
| L. rhamnosus HN001 | 8.38 | 9.06 | 9.36 | 8.29 | 7.47 |
| L. paracasei IM514 | 9.10 | 7.11 | 7.04 | 7.68 | 7.23 |

### 10.3 Conclusion

This Example shows that the particles can provide extended shelf-life under ambient conditions for a variety of bacterial strains.

### 11. Example 11—Lectin Staining for Biofilm Formation

### 11.1 Materials and Methods

Probiotic bead/particles were prepared using Lacticaseibacillus rhamnosus HN001 or Lacticaseibacillus rhamnosus GG (LGG) with the matrix formulations shown in Table 19.

TABLE 19
Matrix formulations.

| | Probiotic | | | | |
|---|---|---|---|---|---|
| | loading | Matrix | Particle | | |
| Sample | (w/w) | composition (w/w) | size | Incubation | Sterilisation |
| 1 | 2.5% HN001 | 100% Ako Comp | ~5 mm | 30 °C | Aseptic dosing |
| | | | bead | | |
| 2 | 2.5% LGG | 100% Ako Comp | ~5 mm | 30 °C | Aseptic dosing |
| | | | bead | | |
| 3 | 2.5% LGG | 99.5% cocoa butter, | ~5 mm | 30 °C | Aseptic dosing |
| | | 0.5% soy lecithin | bead | | |
| 4 | 10% HN001 | 100% Ako Comp | 2-3 mm | 25 °C | Post- |
| | | | pellet | | pasteurisation |
| 5 | 10% LGG | 100% Ako Comp | 2-3 mm | 25 °C | Post- |
| | | | pellet | | pasteurisation |
| 6 | 10% HN001 | 99.5% cocoa butter, | 2-3 mm | 25 °C | Aseptic dosing |
| | | 0.5% soy lecithin | pellet | | |
| 7 | 10% LGG | 99.5% cocoa butter, | 2-3 mm | 25 °C | Post- |
| | | 0.5% soy lecithin | pellet | | pasteurisation |
| 8 | 10% LGG | 99.5% cocoa butter, | 2-3 mm | 25 °C | Aseptic dosing |
| | | 0.5% soy lecithin | pellet | | |

Ako Comp Ingredient (10% Probiotic loading) comprised:
26 g Akosoft 36
60 g Akofine R
4 g Polyglycerol polyricinoleate (PGPR)
10 g Freeze-dried probiotic powder
Total (100 g)

Ako Comp Ingredient (2.5% Probiotic loading) comprised:
28.2 g Akosoft 36
65 g Akofine R
4.3 g Polyglycerol polyricinoleate (PGPR)
2.5 g Freeze-dried probiotic powder
Total (100 g)

Akosoft 36 (hydrogenated coco-glycerides) and Akofine R (hydrogenated vegetable oil) are available from IXOM (Melbourne, Australia).

Samples 1-3 were prepared as described in Example 8, and comparative samples 4-8 were prepared according to the following comparative method:

The fat ingredient(s) and emulsifier (PGPR or soy lecithin) were mixed at 75 °C with continuous stirring until all ingredients were combined. The freeze-dried probiotic powder was added and stirred for 10-20 seconds. The suspension was then pelletised and the pellets ground to a powder.

For all samples, particles/powder were incubated for two weeks in a thick-set UHT yoghurt at the temperature indicated above, as described in Example 1.

Particles were removed, cut in half, and stained with dye mixes 1-4 and visualised by confocal fluorescence microscopy as described in Example 8.

### 11.2 Results

### Exemplary Samples (Samples 1-3)

For sample 1, intense areas of WGA, ConA, and UEA-I fluorescence, extending beyond the cells, were observed. This is consistent with the presence of biofilm.

For sample 2, only small areas of intense WGA fluorescence and no intense regions of ConA fluorescence were observed. Areas of UEA-I fluorescence extending beyond the cells were observed, consistent with the presence of biofilm.

For sample 3, intense WGA fluorescence was observed from the cells, and regions extending beyond the cells were also observed. No intense regions of ConA fluorescence were observed. Intense regions of UEA-I extending beyond the cells were observed. The spread of WGA and UEA-I fluorescence beyond the cells is consistent with biofilm formation.

Overall, samples 1-3 showed evidence of biofilm formation due to WGA and UEA-I fluorescence that extended beyond cell locations as defined by DAPI staining, and lectin staining was not just in the shape of cells. ConA staining consistent with biofilm presence was only observed in particles with HN001 cells (sample 1). ConA is specific for α-mannose and α-glucose, so this difference may be due to differences in biofilm composition between HN001 and LGG.

### Comparative Samples (Samples 4-8)

For sample 4, only small regions of intense WGA fluorescence were observed, no ConA fluorescence was observed, and UEA-I fluorescence more intense than background pooling levels were only observed in small areas around some cell clusters.

Due to sample limitations, sample 5 was not stained with ConA. Intense regions of WGA fluorescence were coincident with the cells and only small areas of intense UEA-I fluorescence were observed.

For sample 6, areas of intense WGA fluorescence were observed that extended beyond the cell, however not all cell clusters exhibited intense WGA fluorescence. No intense fluorescence from ConA was observed. Only some areas of cells appeared to be dyed by UEA-I within sample 6.

For sample 7, WGA fluorescence was unexpectedly concentrated at fat margins that weren't necessarily coincident with cells. ConA staining was generally limited to pooling in non-fat regions. Small regions of intense UEA-I fluorescence about cells were observed.

Sample 8 was identical to sample 7, except that AD sterilisation was used instead of post-pasteurisation. Without post-pasteurisation, the distribution of WGA fluorescence was different. Intense WGA fluorescence was observed, however this did not extend beyond the cells. Areas with large numbers of cells that were not tightly clustered were observed. ConA fluorescence was not observed. UEA-I showed variable stain intensity, however it appeared to be staining cells only, and not extracellular polymeric substances.

Overall, samples 4-8 showed no regions of intense ConA staining except for one very small region of intense ConA fluorescence, but rather yoghurt inclusions into the bead were stained. Only small areas of intense WGA and UEA fluorescence were observed, which did not extend beyond the cells or which were not associated with every cell cluster in the matrix. Samples 4-8 did not show evidence of the extensive biofilm formation seen in samples 1-3.

### 11.3 Conclusion

This Example shows that beads formed using the matrices and processes of the invention showed lectin staining that extended beyond the cells, indicative of extensive biofilm formation.

In contrast, the comparative particles formed using the comparative process generally lacked lectin staining that extended beyond the cells, suggesting that these particles do not contain the same extensive biofilm as the particles of the invention.

### 12. Example 12—Water Vapour Transmission Rate (WVTR)

### 12.1 Materials and Methods

Exemplary particles were prepared as described in Example 9. Comparative particles were also prepared using the comparative method described in Example 11.

WVTR was measured using an aliquot of particle sample (20-60 mg) by weighing in the intrinsic sorption microbalance of a dynamic vapor sorption (DVS) instrument (Surface Measurement Systems, Alperton, Middlesex, UK) at 95% relative humidity. WVTR was measured over a period of 5 minutes, 7 minutes after adjusting the relative humidity to 95% (i.e. from minutes 7 to 12).

WVTR was calculated by using the following equation:

WVTR=dm/(A·dt)

Where dm=the mass of the absorbed water during time dt, and A=total surface area of samples calculated based on particle size measurements. WVTR is reported in units of g/m²/24 h.

Water activity was determined by Aqualab 4TE (Addium Inc, Pullman, WA, United States). Particle size of powder (range of 10 to 1,000 microns) was measured using a stereo microscope followed by analysis using “Image pro plus”.

### 12.2 Results

Water vapour transmission rate (WVTR) and water activity (Aw) was measured for a variety of fat-based matrix formulations (Table 20). All included 0.25% Lacticaseibacillus rhamnosus HN001.

TABLE 20
Surface area to volume ratio (SA/V), water vapour transmission
rate (WVTR) and water activity of particles.

| | | Particle | | WVTR | Water |
|---|---|---|---|---|---|
| Sample | Formulation | diameter | SA/V | (g/m²/24 h) | activity |
| AT03-1 | 74% HCO, 26% Skim milk | 3-5 mm | 1.45 | 98.4 | 0.68 |
| | powder | | | | |
| AT03-2 | 59% HCO, 41% Sucrose | 3-5 mm | 1.45 | 103.8 | 0.55 |
| AT21 | 99.5% CB, 0.5% lecithin | 3-5 mm | 1.45 | 53.7 | 0.49 |
| AT21-1 | 73.5% CB, 26% Skim milk | 3-5 mm | 1.45 | 168.2 | 0.65 |
| | powder, 0.5% soy lecithin | | | | |
| ATX-1 | 86.5% CB, 13.5% Lactose | 3-5 mm | 1.45 | 35.8 | 0.44 |
| AT07-3 | 29.5% PS, 29.5% HCO, 41% | 2-3 mm | 1.98 | 152.8 | 0.49 |
| | Sucrose | | | | |
| AT24 | 100% HPKO | 2-3 mm | 1.83 | 41.7 | 0.43 |
| ATX-2 | 86.5% HCO, 13.5% Lactose | 2-3 mm | 1.77 | 65.1 | 0.42 |

Comparative particles were prepared according to the comparative methods described in Example 11 (“comparative”) and compared to particles of the present invention (“Present inv.”).

Particles containing 2.5% L. rhamnosus HN001 were prepared using the methods described in Example 9, and comparative particles containing 10% L. rhamnosus GG were prepared. Two matrix formulations were used—100% cocoa butter, and 100% Ako Comp. Medium particles had a diameter of approximately 5 mm, and powder particles were ground to a powder.

Particles prepared using the methods of the present invention had a considerably lower WVTR than particles prepared using comparative methods, whether using medium-sized particles or powder, even with similar surface area/volume ratios (Table 21).

TABLE 21
Surface area to volume ratio (SA/V) and water vapour
transmission rate (WVTR) of comparative particles.

| | | | | | WVTR |
|---|---|---|---|---|---|
| Particle diameter | | Technique | Matrix | SA/V | (g/m²/24 h) |
| 2-3 | mm | Present | Cocoa butter | 1.8 | 119.4 |
| | | inv. | Ako Comp | 1.8 | 149 |
| | | Comparative | Cocoa butter | 1.8 | 416 |
| | | | Ako Comp | 1.9 | 381 |
| 10-1000 | μm | Present | Cocoa butter | 48 | 23 |
| | | inv. | Ako Comp | 32 | 62 |
| | | Comparative | Cocoa butter | 33 | 121 |
| | | | Ako Comp | 23 | 196 |

The WVTR was further compared by varying the probiotic used and the particle size. Particles comprising L. rhamnosus GG (LGG) had a higher WVTR than those containing L. rhamnosus HN001, and again particles produced using the comparative methods had a consistently higher WVTR than those produced using the methods of the present invention, even at different particle sizes (Table 22).

TABLE 22
Comparative water vapour transmission rate (WVTR).

| | | Particle | WVTR (g/m²/24 h) | |
|---|---|---|---|---|
| Technique | Composition | diameter | HN001 | LGG |
| Comparative | Cocoa butter | 2-3 mm | 143.2 | 415.50 |
| Present inv. | Cocoa butter | 3-5 mm | 119.4 | 373.92 |
| Comparative | Ako Comp | 2-3 mm | 169.2 | 380.57 |
| Present inv. | Ako Comp | 1-1.6 mm | 148.9 | 133.67 |

### 12.3 Conclusion

This Example shows the WVTR and water activity of particles of the present invention, and comparative particles.

1. A particle comprising a bioactive agent embedded in a biofilm, wherein the biofilm is embedded in a matrix, wherein the matrix has a water vapour transmission rate (WVTR) from 0.1 to 500.

2. The particle of claim 1, wherein the matrix comprises at least 6% by weight of one or more lipids, preferably 10-100% by weight, more preferably 30-100%.

3. The particle of claim 1 or 2, wherein the matrix has a water vapour transmission rate (WVTR) from 0.1 to 10, preferably from 1 to 5.

4. The particle of any one of claims 1 to 3, wherein the bioactive agent comprises a first microorganism, preferably a probiotic microorganism.

5. The particle of claim 4, wherein the biofilm is produced by the first microorganism.

6. The particle of claim 4 or 5, wherein the first microorganism is a Bacillus, Bifidobacterium, Enterococcus, Lacticaseibacillus, Lactiplantibacillus, Lactobacillus, Lactococcus, Ligilactobacillus, Limosilactobacillus, Lentilactobacillus, Saccharomyces, or Streptococcus; preferably wherein the first microorganism is selected from the group consisting of Bacillus coagulans, Bifidobacterium animalis, Bifidobacterium breve, Bifidobacterium bifidum, Bifidobacterium longum, Lacticaseibacillus casei, Lacticaseibacillus paracasei, Lacticaseibacillus rhamnosus, Lactiplantibacillus plantarum subsp. plantarum, Lactobacillus acidophilus, Lactobacillus delbrueckii, Lactobacillus gasseri, Lactococcus lactis, Lactococcus lactis subsp. lactis, Lactococcus lactis subsp. lactis biovar diacetylactis, Leuconostoc pseudomesenteroides, Ligilactobacillus salivarius, Limosilactobacillus reuteri, Saccharomyces boulardii, and Streptococcus thermophilus; more preferably wherein the first microorganism is Lacticaseibacillus rhamnosus HN001.

7. The particle of any one of claims 4 to 6, comprising the first microorganism in an amount of from 10⁵ to 10¹² CFU/g of the particle, preferably from 10⁶ to 10¹⁰, more preferably from 10⁷ to 10⁹, most preferably about 10⁸ CFU/g.

8. The particle of any one of claims 4 to 7, further comprising a second microorganism that is different from the first microorganism, preferably wherein the second microorganism is a probiotic microorganism.

9. The particle of any one of claims 1 to 8, further comprising a non-microbial bioactive agent, preferably a vitamin, a prebiotic, a postbiotic, and/or a human milk oligosaccharide.

10. The particle of any one of claims 1 to 9, wherein the biofilm is characterised by peaks in a principal components analysis of Raman spectroscopy data at 360, 430, and/or 530 cm⁻¹.

11. The particle of any one of claims 1 to 10, wherein the biofilm is capable of being stained by concanavalin A, wheat germ agglutinin, and/or Ulex europaeus agglutinin.

12. The particle of any one of claims 1 to 11, wherein the particle maintains a moisture content of from 1% to 60% by weight within the matrix when included in another foodstuff with a water activity of from 0.90 to 1.00 for a period of 2 weeks, preferably a moisture content of from 10% to 60%, more preferably a moisture content of from 20% to 60%.

13. The particle of any one of claims 2 to 12, wherein the one or more lipids:
a. are solid at 20 °C, 25 °C, and/or 30 °C;
b. have a melting temperature of 50 °C or lower, preferably from 30 °C to 50 °C;
c. comprise, or consist of, triglycerides;
d. comprise C12-C20 fatty acids or salts or esters thereof; or
e. any combination of any two or more of (a) to (d).

14. The particle of any one of claims 2 to 13, wherein the one or more lipids comprise one or more fatty acids and/or salts and/or esters thereof, and wherein:
a. at least 1% by weight of the fatty acids and/or salts and/or esters thereof have a carbon chain length of 14 or less, preferably from 1% to 80% by weight; and/or
b. at least 30% by weight of the fatty acids and/or salts and/or esters thereof have a carbon chain length of 16 or less, preferably at least 70%, preferably from 30% to 90% by weight, more preferably from 70% to 90%.

15. The particle of any one of claims 2 to 14, wherein at least 50% by weight of the one or more lipids are saturated fats.

16. The particle of any one of claims 2 to 15, wherein the one or more lipids comprise, or consist of, fully hydrogenated palm kernel stearin, palm stearin, fully hydrogenated coconut oil, hydrogenated palm kernel oil, or a mixture of any two or more of these.

17. The particle of any one of claims 1 to 16, wherein the matrix comprises at least 10% by weight of fully hydrogenated coconut oil, preferably at least 20%, more preferably at least 30%.

18. The particle of any one of claims 1 to 17, wherein the particle has a particle size of from 50 μm to 10 mm.

19. The particle of any one of claims 1 to 18, further comprising at least one coating layer.

20. A food or beverage product comprising the particle of any previous claim.

21. The food or beverage product of claim 20, wherein the food or beverage product comprises:
a. a protein powder, a protein shake, a protein shot, a protein gel, or a sports nutritional formulation,
b. a UHT beverage, a UHT smoothie,
c. an infant formula, a follow-on formula, a growing-up formula, a paediatric formula, a human milk fortifier, a children's food or drink, a maternal supplement, or a maternal nutritional formulation,
d. a gel such as a heat-set gel, a sauce, a spread, a jam, a jelly, or a honey,
e. an acid-set gel, an ambient stable yoghurt, a stirred yoghurt, a set yoghurt, or a drinking yoghurt,
f. a neutral beverage, an acidic beverage, a water, a juice, a milk, a smoothie, a shake, a shot, an alcoholic beverage, a soft drink, a kombucha, a kefir, or a ready-to-mix powder,
g. a bar, a ball, a cake, a cookie, a muffin, or a bakery good,
h. a medical food, a soup, a dessert, a pudding, a custard, an enteral formulation, a formulation for senior or aged populations, a table, a capsule, or a supplement,
i. a confection, a gummy, a candy, a chocolate, a fudge, a truffle, a chewing gum, a frozen dessert, or an ice cream,
j. flavouring, a topping, or a baking ingredient,
k. a cream, a cheese, or a butter, or
l. a livestock food or a pet food.

22. The food or beverage product of claim 20 or claim 21, wherein the food or beverage product has a water activity of at least 0.2, 0.4, 0.6, 0.8, or 0.9.

23. The food or beverage product of any one of claims 20 to 22, wherein the food or beverage product is stable at 30 °C for at least 30 days, preferably for at least 60 days, more preferably for at least 6 months.

24. The food or beverage product of any one of claims 20 to 23, wherein the bioactive agent comprises a microorganism, and wherein the degradation rate of the microorganism measured over 12 months at 30 °C is less than 7 log CFU/g/y, preferably less than 6, 5, 4, 3, 2, or 1 log CFU/g/y.

25. A method of increasing the stability of a bioactive agent, the method comprising the steps of:
a. embedding the bioactive agent in a biofilm,
b. embedding the biofilm in a matrix having a water vapour transmission rate (WVTR) from 0.1 to 500, and
c. forming the matrix into particles.

26. A method of increasing the stability of a bioactive agent, the method comprising the steps of:
a. embedding the bioactive agent in a matrix having a water vapour transmission rate (WVTR) from 0.1 to 500,
b. inducing the bioactive agent to produce a biofilm, and
c. forming the matrix into particles,
wherein steps b) and c) can be in any order.

27. The method of claim 25 or 26, wherein the matrix comprises at least 6% by weight of one or more lipids.

28. The method of any one of claims 25 to 27, wherein the bioactive agent comprises a first microorganism.

29. The method of claim 28, wherein the biofilm is produced by the first microorganism.

30. The method of any one of claims 25 to 29, further comprising the step of coating the particles in at least one coating layer.

31. The method of any one of claims 25 to 30, wherein the particles comprise the particle of any one of claims 1 to 19.

32. The method of any one of claims 25 to 31, further comprising the steps of:
d. combining the particles of step c with a medium having a water activity of at least 0.5, preferably a UHT-treated yoghurt, and
e. incubating the medium for between 3 and 30 days.

33. A method of producing a bioactive agent supplemented food or beverage product, the method comprising the method of claim 32, and further comprising the steps of:
f. harvesting the particles from the medium of step e, and
g. combining the harvested particles with a food or beverage product, thus obtaining the bioactive agent supplemented food or beverage product.

34. A method of producing a bioactive agent supplemented food or beverage product, the method comprising the method of any one of claims 25 to 32, and further comprising the step of combining the particles with a food or beverage product, thus obtaining the bioactive agent supplemented food or beverage product.

35. The method of claim 33 or claim 34, wherein the bioactive agent supplemented food or beverage product is a food or beverage product according to any one of claims 20 to 24.
